(19) United States
(12) Patent Application Publication
(10) Pub. No.: US 2025/0268921 A1
(43) Pub. Date: Aug. 28, 2025

(54) OPTHALMIC FORMULATIONS AND USES THEREOF

(71) Applicant: TelomEYE Pharmaceutical Corporation, Toronto (CA)

(72) Inventors: Iskender Alkin SOLMAZ, Istanbul (TR); Isilay KAVADARLI, Istanbul (TR); Canan Sevimli GUR, Izmir (TR); Murat Kubilay TURKMEN, Burlington (CA)

(73) Assignee: TelomEYE Pharmaceutical Corporation, Toronto (CA)

(21) Appl. No.: 19/206,938

(22) Filed: May 13, 2025

Related U.S. Application Data
(60) Provisional application No. 63/646,277, filed on May 13, 2024.

(30) Foreign Application Priority Data
Jul. 31, 2018 (TR) 2018/11088

(51) Int. Cl. A61K 31/58; A61K 9/00; A61K 47/18; A61K 47/20; A61K 47/38; A61P 27/02

(52) U.S. Cl. CPC A61K 31/58; A61K 9/0048; A61K 47/183; A61K 47/186; A61K 47/20; A61K 47/38; A61P 27/02

(57) ABSTRACT

Disclosed herein are ophthalmic formulations and eye drop formulations for treatment, prevention and/or reducing a risk of age-related conditions, including corneal endothelial dysfunction, particularly Fuchs' endothelial corneal dystrophy (FECD) or corneal edema. The formulations comprise an active agent for increasing telomerase activity. Also provided are methods for preparing said formulations.

### CROSS-REFERENCE TO RELATED APPLICATIONS

This application is a Non-Provisional U.S. Application, claiming benefit and priority to Provisional Application 63/646,277, filed on May 13, 2024, which is a continuation-in-part (CIP) application of U.S. patent application Ser. No. 17/163,119, filed on Jan. 29, 2021, which claims benefit of and priority under 35 U.S.C § 120 of international patent application PCT/US2019/042368, filed Jul. 18, 2019, which claims benefit of priority under 35 U.S.C. § 119(a) of Turkish Application No. 2018/11088, filed Jul. 31, 2018, the entirety of which is hereby incorporated by reference.

### TECHNICAL FIELD

The invention relates to ophthalmic formulations and eye health. More specifically the present invention is directed to ophthalmic formulations for activating telomerase and methods for increasing the viability of corneal tissues or cells by activating telomerase.

### BACKGROUND

The eye is one of the first sensory organs to experience the signs of aging. While natural age-related changes may have direct impact on vision, age related illnesses unrelated with the eye can also impact the eye. Astigmatism, myopia, hypermetropia, presbyopia, hordeolum, glaucoma, amblyopia, cataract, diplopia, nyctalopia, eye pain, red eye, eye allergies, xerophthalmia, tearing, swollen eyelid and age-related macular degeneration are common ophthalmic diseases. There are several eye diseases that start without any early symptoms. For example, the pupil gets smaller and its response to light weakens with age leading to difficulties in seeing in dim light and delayed dark-light adaptation. Flexibility of the crystalline lens also diminishes with age resulting in presbyopia.

Aging also affects the fibers attached to the ciliary body, leading to impaired refractive capacity. The transparent eye lens also starts to turn yellow with age, reducing transmission, increasing the amount of blue light absorbed, and increasing scattering thereby impairing color perception and contrast. Dry eye (keratoconjunctivitis sicca) is another ailment caused by aging associated tear hyperosmolarity and epithelial cell damage leading to aqueous layer failure.

The most important reason for central vision loss in Americans over the age of 50is age related macular degeneration. Prevalence of this disease is roughly 85 to 90% for dry type and 10 to 15% for neovascular type. Aging related changes on the macula, which affects the outer retina, the retina pigment epithelium (RPE), the Bruch membrane and choriocapillaries cannot always be diagnosed clinically. Glaucoma is another eye disease that is characterized with high levels of intraocular pressure that impairs the optic nerve head. The two primary types of glaucoma are congenital and acquired glaucoma. Approximately 2% of people over the age of 40 and 10% over the age of 80 suffer from acquired glaucoma.

The telomere is a special heterochromatin structure present on the edges of eukaryotic chromosomes. Telomeres might have a length from 1-50 kb depending on type of cell and genetic history. Human telomeres are 10-15 kb in reproductive cells but are shorter in some somatic cells. Telomeres protect chromosome edges from random double chain DNA fragmentation thereby avoiding fusion of chromosomes. Besides this physical protection of chromosomes, eukaryotic telomeres have important cellular functions such as chromatin organization, chromosome replication and cell reproduction. Telomere length in DNA is reduced by about 50 to 150 base pairs for each cell division and this shortening eventually restricts the number of cell divisions a cell can make leading to accelerated or delayed control of cell aging. The most common method for maintaining telomere length is increasing telomerase activity. The ability of telomerase to protect cell demise has led to an interest in developing telomerase inhibitors to treat cancer. In contrast, telomerase activators are expected to slow down the aging process and hence may be relevant to the treatment of aging-related eye diseases. While there have been some efforts to increase telomerase activity by delivering telomerase into cells, the art is deficient in formulations that may be used to slow the aging process to treat eye conditions. The present invention fulfills this longstanding need and desire in the art.

### SUMMARY

The present invention is directed to an ophthalmic formulation. The ophthalmic formulation comprises, in an isotonic solution, a telomerase activator, an antimicrobial agent, a chelator and at least one excipient.

The present invention also is directed to an eye drops formulation. The eye drops formulation comprises cycloastragenol at a concentration of about 3 ng/ml to about 500 ng/ml in an isotonic saline solution containing an antimicrobial agent, a chelator and an excipient.

The present invention is directed further to a method for reducing the incidence of an age-related eye condition in a subject. In the method an amount of the ophthalmic formulation described herein that is effective to increase an activity of telomerase in the corneal tissue is administered to one or both eyes of the subject thereby reducing the incidence of age-related eye diseases.

The present invention is directed further still to a method for increasing viability of cells in the corneal tissue of a subject. In the method, both eyes of the subject are contacted with an amount of the ophthalmic formulation described herein.

Other and further aspects, features, and advantages of the present invention will be apparent from the following description of the presently preferred embodiments of the invention. These embodiments are given for the purpose of disclosure.

### Selected Definitions and Nomenclature

The term “about” is used in conjunction with numeric values to include normal variations in measurements as expected by persons skilled in the art, and is understood to have the same meaning as “approximately” and to cover a typical margin of error, such as ±15%, ±10%, ±5%, ±1%, ±0.5%, or even ±0.1% of the stated value. The term “about” also encompasses amounts that differ due to different equilibrium conditions for a composition resulting from a particular initial composition. Whether or not modified by the term “about,” the claims include equivalents to the quantities.

It should be noted that, as used in this specification and the appended claims, the singular forms “a,” “an,” and “the” include plural referents unless the content clearly dictates otherwise. Thus, for example, reference to a composition containing “a compound” includes having two or more compounds that are either the same or different from each other. It should also be noted that the term “or” is generally employed in its sense including “and/or” unless the content clearly dictates otherwise. As used herein, “and/or” refers to and encompasses any and all possible combinations of one or more of the associated listed items, as well as the lack of combinations when interpreted in the alternative (“or”).

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “comprise,” “comprises,” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 1%, 5%, 10%, 15%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 1%, 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5% or even 1%) detectable activity or amount.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

As used throughout this description, and in the claims, a list of items joined by the term “at least one of” or “one or more of” can mean any combination of the listed terms. For example, the phrase “at least one of X, Y or Z” can mean X; Y; Z; X and Y; X and Z; Y and Z; or X, Y and Z

“A”, “an”, and “the” as used herein refers to both singular and plural referents unless the context clearly dictates otherwise.

As used herein, the term “ophthalmic formulation” or “formulation” are interchangeable and includes eye drops formulation which comprises an indicated concentration of a telomerase activator as active agent. A “placebo” or “placebo formulation” is a formulation without the active agent.

As used herein, the term “subject” refers to any recipient of the ophthalmic formulation whether it contains an active agent or is a placebo. The subject is a mammal, preferably, a human.

As used herein, the term “condition” or “eye condition” are interchangeable and refers to any pathophysiological disorder or disease or change in vision or visual acuity of the eye occurring as a result of aging.

As used herein, the term “trauma” or “ocular trauma” are used interchangeably and refer to conditions which can result in endothelial dysfunction from surgical trauma, such as intraocular surgeries, penetrating or blunt physical injury related trauma, infections, or chemical injuries, such as caused by certain ophthalmic medications, including adverse effects from preservatives within ophthalmic medications, or acids and bases in cleaning solutions.

### BRIEF DESCRIPTION OF THE DRAWINGS

FIGS. 1A-1B show an in vitro viability analysis in corneal endothelial cells. FIG. 1A shows a bar chart representation of cell viability in corneal endothelial cells treated with the indicated concentrations of Formulation eye drop formulation for 48 hours. FIG. 1B shows a Tendency/Regression Line and Tendency Equation of the cell viability data shown in FIG. 1A. IC₅₀ value was calculated from this graph to be 40.2 μg/ml.

FIGS. 2A-2B shows graphical representation and photomicrographs for the effect Of Formulation eye drop formulation containing various concentrations of the active compound cycloastragenol in 1% DMSO, on viability of corneal endothelial cells. FIG. 2A is a cell viability analysis in corneal endothelial cells treated with the indicated concentrations of cycloastragenol in 1% DMSO. Data were analyzed 48 hours after treatment. FIG. 2B shows representative photomicrographs for each of the cycloastragenol concentrations in FIG. 2A.

FIGS. 3A-3B show a graphical representation and photomicrographs of the effect of the Formulation eye drop formulation containing various concentrations of the active compound cycloastragenol in 0.2% DMSO, on viability of corneal endothelial cells. FIG. 3A is a cell viability analysis in corneal endothelial cells treated with the indicated concentrations of cycloastragenol in 1% DMSO. Data were analyzed 48 hours after treatment. FIG. 3B shows representative photomicrographs for each of the cycloastragenol concentrations in FIG. 3A.

FIGS. 4A-4B show in vitro telomerase activity in corneal endothelial cells treated with the indicated concentrations of cycloastragenol in the Formulation eye drop formulation. FIG. 4A shows the effect of cycloastragenol concentration on telomerase activity in the corneal endothelial cell line HCEC-B4G12 treated for 30 hours. Untreated (NC) and placebo (Formulation eye drop formulation with 1% DMSO carrier but no cycloastragenol). Data were mean±standard deviation obtained 3 independent experiments, each repeating thrice. FIG. 4B is a line plot of the data shown in FIG. 4A.

FIGS. 5A-5B show time dependent changes to relative cell density (CD) in corneal layer endothelial cells of rabbits treated with Formulation eye drop formulation. FIG. 5A shows the effect of cycloastragenol in the Formulation eye drop formulation on cell density at the end of months 0, 1, 3 and 6. Untreated (negative control), placebo (eye drop Formulation with DMSO carrier but no cycloastragenol) are compared with groups B, C and D containing cycloastragenol at 25, 100 and 500 ng/ml. Results shown are normalized to the 0-month data. FIG. 5B is a line plot of the data shown in FIG. 5A.

FIGS. 6A-6B show time dependent changes to relative cell density (CD) in corneal layer endothelial cells of rabbits treated with Formulation eye drop formulation. FIG. 6A shows the effect of cycloastragenol in the Formulation eye drop formulation on cell density at the end of months 0, 1, 3 and 6. Untreated (negative control), placebo (Formulation eye drop formulation with DMSO carrier but no cycloastragenol) are compared with groups B, C and D containing cycloastragenol at 25, 100 and 500 ng/ml. Results shown are normalized to the negative control group. FIG. 6B is a line plot of the data shown in FIG. 6A.

FIGS. 7A-7B show time dependent changes to relative cell density (CD) in corneal layer endothelial cells of rabbits treated with Formulation eye drop formulation using the placebo for normalization. FIG. 7A shows the effect of cycloastragenol in the Formulation eye drop formulation on cell density at the end of months 0, 1, 3 and 6. Untreated (negative control), placebo (Formulation eye drop formulation with DMSO carrier but no cycloastragenol) are compared with groups B, C and D containing cycloastragenol at 25, 100 and 500 ng/ml. FIG. 7B is a line plot of the data shown in FIG. 7A.

FIGS. 8A-8E are representative histology sections showing Mallory-Azan (left panel) and Hematoxylin-Eosin (H&E, right panel) staining of the corneal layer in the various treatment groups described in FIG. 7. FIG. 8A shows Mallory-Azan and H&E staining for negative control group. FIG. 8B shows Mallory-Azan and H&E staining for placebo group. FIG. 8C shows Mallory-Azan and H&E staining for Group B (25 ng/ml). FIG. 8D shows Mallory-Azan and H&E staining for Group C (100 ng/ml). FIG. 8E shows Mallory-Azan and H&E staining for Group D (500 ng/ml).

FIG. 9 shows the effect of an eye drop formulation containing cycloastragenol on telomere length in the corneal endothelial cells of treated rabbits.

FIG. 10 is a histogram showing the distribution of telomere lengths in a representative sample. Bars represent the relative frequency for every particular fluorescence intensity normalized (X axis). The 20th percentile (red bars) indicates the particular length below which 20% of the telomeres have been observed. The median (MTL) and average (ATL) telomere length are also indicated in the histogram. This histogram also allows for the analysis of telomere length variability.

FIGS. 11A-11E show representative images used for telomere length analysis in corneal endothelial cells from control and treated rabbits. FIG. 11A is representative image used for telomere length analysis in corneal endothelial cells in rabbits treated with Group A FIG. 11B is representative image used for telomere length analysis in corneal endothelial cells in rabbits treated with Group B. FIG. 11C is representative image used for telomere length analysis in corneal endothelial cells in rabbits treated with Group C. FIG. 11D is representative image used for telomere length analysis in corneal endothelial cells in rabbits treated with Group D. FIG. 11E is representative image used for telomere length analysis in corneal endothelial cells in rabbits treated with Group N.

FIG. 12 depicts graphical results for cornea endothelial cell count related to the administration of 4 μg/50 μl eye drop formulation.

FIG. 13 depicts graphical results for cornea endothelial cell count related to the administration of 8 μg/50 μl eye drop formulation.

FIG. 14 depicts graphical results for cornea endothelial cell count related to the administration of 12 μg/50 μl eye drop formulation.

FIG. 15 depicts a graph of time-dependent corneal endothelial cell count changes in all dose groups treated with eye drop formulations, in accordance with embodiments disclosed herein.

FIG. 16A shows images of histology sections with Mayer's hematoxylin counterstain. FIG. 16A shows telomerase activity in cornea epithelial and endothelial cells (top left) in 80 μg/ml TAED017 eye drop group when compared to non-treated placebo control group cornea tissue (top right). Prominent and dense tert antigen immunostaining (telomerase activity) in the ciliary processes of TAED017 treated iris tissue (bottom left) can be seen, in contrast to weak and very limited expression in non-treated control iris tissue (bottom right).

FIG. 16B shows images of histology sections similar to FIG. 16A, but for a dosage of 160 μg/ml TAED017 eye drop.

FIG. 16C shows images of histology sections similar to FIG. 16A, but for a dosage of 240 μg/ml TAED017 eye drop.

FIG. 17 illustrates dose-dependent increase of telomerase reverse transcriptase (TERT) protein expression in ocular tissues following TAED017 treatment. Significantly higher TERT protein expression was observed in the cornea and iris tissues of TAED017-treated left eyes compared to untreated tissues. TERT expression levels, quantified by Allred score, are shown on the Y-axis, plotted against escalating dosages of TAED017 on the X-axis.

F

IG. 18 illustrates a graph showing percentage of telomere length increases in treated eyes (L) versus control (untreated eyes, R), with increasing dosages of telomerase activator. Treatment with TAED017 results in Corneal Endothelial Cells (CEC) telomeres that are longer than those from untreated eyes (L: treated eyes, R: non-treated-control eyes). (Cy3=Fluorescence signal of telomere probe, Hoescht=fluorescence of the nuclei, Average R=Average of the control eye).

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative bases for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical application. Various combinations and modifications of the features consistent with the teachings of this disclosure, however, could be desired for particular applications or implementations.

In one embodiment of the present invention there is provided an ophthalmic formulation comprising in an isotonic saline solution a telomerase activator; an antimicrobial agent; a chelator; and at least one excipient. In this embodiment the telomerase activator may be cycloastragenol. Also in this embodiment the cycloastragenol concentration in the isotonic saline solution is about 3 ng/ml to about 500 ng/ml. In one aspect of this embodiment the cycloastragenol concentration in the isotonic saline solution is about 25 ng/ml. In another aspect the cycloastragenol concentration in the isotonic saline solution is about 100 ng/ml. In yet another aspect the cycloastragenol concentration in the isotonic saline solution is about 500 ng/ml.

Also in this embodiment the antimicrobial agent may be benzalkonium chloride at a concentration in the isotonic saline solution of about 0.005% (w/v) to about 0.02% (w/v). In a representative example the antimicrobial agent is benzalkonium chloride at a concentration in the isotonic saline solution of 0.01% (w/v).

In addition in this embodiment the chelator may be ethylenediaminetetraacetic acid, disodium salt at a concentration in the isotonic saline solution of about 0.005% (w/v) to about 0.02% (w/v). In a representative example the chelator is ethylenediaminetetraacetic acid, disodium salt at a concentration in the isotonic saline solution of about 0.01% (w/v).

Further in this embodiment the excipient is hydroxypropyl methyl cellulose, polyvinylpyrrolidone, ethanol, or dimethyl sulfoxide or a combination thereof. Further still the excipient concentration in the isotonic saline solution may be about 0.05% (w/v) to about 0.9% (w/v). In one aspect of this further embodiment the excipient concentration in the isotonic saline solution is about 0.05% (v/v) to about 1% (v/v). In another aspect the excipient is hydroxypropyl methyl cellulose at a concentration in the isotonic saline solution of about 0.125% (w/v).

In another embodiment of the present invention there is provided an eye drops formulation, comprising cycloastragenol at a concentration of about 3 ng/ml to about 500 ng/ml in an isotonic saline solution containing an antimicrobial agent, a chelator and an excipient.

In this embodiment the cycloastragenol concentration in the isotonic saline solution may be selected from the group consisting of 25 ng/ml, 100 ng/ml and 500 ng/ml.

Also in this embodiment the antimicrobial agent contained in the isotonic saline solution may be benzalkonium chloride at a concentration of about 0.01% (w/v).

In addition the chelator contained in the isotonic saline solution may be ethylenediaminetetraacetic acid, disodium salt at a concentration in the isotonic saline solution of about 0.01% (w/v).

Further the excipient contained in the isotonic saline solution is at least one of hydroxypropyl methyl cellulose, polyvinylpyrrolidone, ethanol, or dimethyl sulfoxide at a concentration of about 0.05% (w/v) to about 0.9% (w/v) or is about 0.05% (v/v) to about 1% (v/v). In a representative example the excipient contained in the isotonic saline solution is hydroxypropyl methyl cellulose at a concentration of about 0.125% (w/v).

In yet another embodiment of the present invention there is provided a method for reducing the incidence of an age-related eye condition in an subject comprising administering to one or both eyes of the subject an amount of the ophthalmic formulation as described supra effective to increase an activity of telomerase in the corneal tissue thereby reducing the incidence of age-related eye condition.

In this embodiment the ophthalmic formulation may be administered topically. Also in the embodiment the ophthalmic formulation may be administered at least once daily over a period of about one month to about twelve months, or a period lasting longer than twelve months. In addition the age-related eye condition may be macular degeneration, cataract, presbyopia, or glaucoma, or age related dry eye, or a combination thereof.

In yet another embodiment of the present invention there is provided a method for increasing viability of cells in the corneal tissue of a subject, comprising contacting both eyes of the subject with an amount of the ophthalmic formulation as described supra

In this embodiment the contacting step may comprise topically contacting one or both of the eyes with the ophthalmic formulation. Also in this embodiment, the eye may be contacted with the ophthalmic formulation at least once daily. In addition, the subject may have an age-related eye condition. Particularly, the age-related eye condition may be macular degeneration, cataract, presbyopia, or glaucoma, or age related dry eye or a combination thereof.

Provided herein are ophthalmic formulations effective for increasing, telomerase activity, increasing telomere length and increasing the viability of cells in corneal tissue. The ophthalmic formulations generally are formulated in a saline solution, preferably, an isotonic saline solution and contain a telomerase activator as the active compound, an antimicrobial agent, a chelator, and at least one excipient, for example, any suitable physiologically acceptable excipient. The ophthalmic formulations may be eye drops.

The telomerase activator may be any compound that is known in the art to prevent loss of telomeres from the ends of the chromosomes or to promote addition of telomere sequences to the ends of the chromosomes. Alternatively, the telomerase activator may a combination of these compounds in the same or varying concentrations. In a non-limiting example, the telomerase activator is cycloastragenol at a concentration of about of about 3 ng/ml to about 500 ng/ml in the isotonic saline solution. In other embodiments the telomerase activator can include astragaloside I, astragaloside II, astragaloside IV, astragaloside VII (AST VII), cyclocephaloside I (CCI), cyclocanthoside E (CCE), andrographolide, resveratrol, epitalon, estrogen, omega-3 fatty acids, and their derivatives or combinations thereof.

The antimicrobial agent protects the formulation from bacterial and/or fungal growth. The antimicrobial agent or a combination of antimicrobial agents may be used at any suitable concentration that would prevent growth of microbes and one of ordinary skill in this art would be readily able to discern the desired concentration that would prevent microbial growth. A non-limiting example of an antimicrobial agent is benzalkonium chloride at a concentration of about 0.005% (w/v) to about 0.02% (w/v), for example, about 0.01% (w/v), in the isotonic saline solution. Alternatively, the ophthalmic formulation containing the telomerase activator may be combined with other ophthalmic formulations in clinical use for the treatment of eye diseases including, but not limited to, formulations for glaucoma and age-related macular degeneration (AMD), or age related dry eye.

The chelator increases shelf life by improving stability of the formulation and may be used at any suitable concentration. In non-limiting examples, the chelator is an ethylenediamine salt, a ethylenediaminetetraacetic acid disodium salt and an ethyleneglycoltetraacetic acid salt. Suitable concentrations for the chelator are about 0.005% (w/v) to about 0.02% (w/v), for example, about 0.01% (w/v), in the isotonic saline. The chelator may be disodium ethylenediaminetetraacetic acid disodium salt.

Each excipient is “physiologically acceptable” in the sense of not being injurious to biological cells and the subject, of being compatible with the other components in the formulation, and of not limiting the efficacy of the telomerase activator. The excipient may comprise a single compound or a combination of compounds in varying proportions, each ranging in concentration from about 0.05% (w/v) to about 0.9% (w/v) or from about 0.05% (v/v) to about 1% (v/v). In non-limiting examples, the excipient is at least one of hydroxypropyl methyl cellulose, polyvinylpyrrolidone, ethanol, or dimethyl sulfoxide. The excipient may be hydroxypropyl methyl cellulose at a concentration of about 0.125% (w/v). Alternatively, the excipients may be hydroxypropyl methyl cellulose and dimethylsulfoxide. In further embodiments, excipients may include, hydroxypropyl methylcellulose (HPMC), carbomer (carbopol-polyacrylic acid derivatives), sodium hyaluronat (hyaluronic acid), polyvinyl alcohol (PVA), polyethylene glycol (PEG) or propylene glycol (PPG), carboxymethylcellulose sodium-CMC, xanthan gum or combinations thereof.

Also provided are methods that reduce the incidence of an age-related eye condition and/or increase the viability of cells in corneal tissue that utilize the ophthalmic formulations or eye drops described herein. The formulations or eye drops are administered to one or both eyes of a subject or the eyes are contacted with the formulations or eye drops. The age-related conditions of the eyes is any clinical ophthalmic condition that is caused due to decreased viability and/or reduced telomere length in cells of the corneal tissue or that occurs naturally as one ages. These conditions may include, but are not limited to, macular degeneration, cataract, presbyopia and glaucoma, age related dry eye, or a combination thereof.

Routes for administering or contacting the eye(s) are well-known routes applicable to a particular condition or to reduce or delay the incidence or onset of a particular condition and/or useful during an ophthalmic surgical procedure. For example, useful routes are, but not limited to, topical, intraocular, intravitreal, or system. One of skill in this art is well able to determine the most appropriate delivery method in view of the condition of the eyes.

Generally, one skilled in this art is able to determine the dose and dosage routine of the formulations depending on the health of the subject and the condition affecting the eyes. The formulations may be administered or the eye(s) contacted with the formulations or eye drops once, twice, or more times daily depending on the type and/or progression of the condition affecting the eye(s). This routine may occur for about one month to about 12 months, or for a period longer than 12 months. Alternatively, the formulation may be utilized until the condition has improved or until the condition has resolved in the subject.

Also disclosed herein are embodiments for an ophthalmic formulation for treating, preventing and/or reducing a risk of a corneal endothelial dysfunction, wherein the ophthalmological formulation comprises a therapeutically effective amount of a telomerase activator. The ophthalmic formulation comprises, in an isotonic, hypertonic or hypotonic solution, a telomerase activator, an antimicrobial agent, a chelator and at least one excipient.

The formulations disclosed herein can further be used in the prevention, or treatment of other ocular diseases or traumas. Endothelial decompensation, which manifests as corneal opacity, arises from a variety of primary and secondary causes. The most common primary etiology is Fuchs' endothelial corneal dystrophy (FECD). Other inherited conditions like posterior polymorphous corneal dystrophy (PPCD), congenital hereditary endothelial dystrophy (CHED), and Iridocorneal Endothelial (ICE) syndrome also contribute to endothelial dysfunction. Secondary causes often involve surgical trauma, particularly from cataract extraction and intraocular lens (IOL) implantation, leading to pseudophakic bullous keratopathy (PBK)-the most frequent secondary cause. Chemical injuries from preservatives in ophthalmic medications or improper rinsing of surgical instruments can further damage endothelial cells. Trauma, whether blunt or penetrating, and infections such as herpes simplex endotheliitis also compromise endothelial function. Additionally, glaucoma and its treatments (e.g., medications with toxic preservatives, surgical interventions like trabeculectomy, or previous failed corneal grafts) exacerbate endothelial cell loss. Additionally, materials used in pars plana vitrectomy, such as silicone, migrate to the anterior chamber over time and lead to endothelial decompensation.

In one embodiment, the corneal endothelial dysfunction is Fuchs' endothelial corneal dystrophy (FECD), corneal edema, bullous keratopathy (PBK), acute or chronic corneal endothelial cell (CEC) loss, corneal degeneration, corneal melting, or corneal ectasia, or any combination thereof.

In one embodiment, the corneal endothelial dysfunction is Fuchs' endothelial corneal dystrophy (FECD). The cornea is composed of three cellular layers: the epithelium, the stroma and the endothelium. To maintain its transparency, the corneal stroma needs to be partially dehydrated to ensure proper collagen fibrils arrangement. Fuchs endothelial corneal dystrophy (FECD) is a corneal pathology characterized by an accelerated loss of endothelial cells and abnormal extracellular deposition. When endothelial cell loss reaches a point where the endothelium can no longer perform its role, corneal edema develops, causing vision loss. Presently, subjects diagnosed with FECD only have the option of a corneal transplant, since there is no other effective treatment that prevents blindness. Recent studies show evidence of shorter telomere length present in subjects diagnosed with FECD.

Therefore, the present invention proposes a treatment, prevention or reduction of risk, or slowing of progression of FECD, by use of an ophthalmic formulation which comprises the telomerase activator disclosed herein. In one embodiment, the ophthalmic formation comprises therapeutically effective amount of cycloastragenol.

In other embodiments, the telomerase activator can be astragaloside I, astragaloside II, astragaloside IV, astragaloside VII (AST VII), cyclocephaloside I (CCI), cyclocanthoside E (CCE), andrographolide, resveratrol, epitalon, estrogen and omega-3 fatty acids, their derivatives or combinations thereof.

In a further embodiment, the corneal endothelial dysfunction is corneal edema. Corneal edema is characterized by swelling in the cornea that can occur after injury or an infection. Corneal edema can also occur due to inherited conditions, such as Fuchs's dystrophy, wherein the corneal edema is the result of progressed state of Fuchs'.

In one embodiment, the ophthalmic formulation for treating, preventing or reducing the risk of the corneal endothelial dysfunction disclosed above, comprises the active ingredient, the telomerase activator, in a concentration of about 0.3 μg/ml-370 μg/ml. In a further embodiment, the telomerase activator is present in a concentration of 0.5 μg/ml-350 μg/ml, 10 μg/ml-320 μg/ml, 20 μg/ml-290 μg/ml, 30 μg/ml-270, 40 μg/ml-260 μg/ml, 50 μg/ml-250 μg/ml, or 80 μg/ml to about 240 μg/ml or any range or value therebetween.

In further embodiments, the ophthalmic formulation can further incorporate an antimicrobial agent, a chelator and an excipient, as described and detailed above in prior embodiments. In embodiments, the antimicrobial agent contained in the ophthalmic formulation may be benzalkonium chloride at a concentration of about 0.01% (w/v). The chelator may be ethylenediaminetetraacetic acid disodium salt at a concentration of about 0.01% (w/v). Further chelators which may used are diethylene triamine pentacetic acid (DTPA), ethylenediamine tetramethylene phosphonic acid, citric acid or citrates, Deferoxamine, Deferasirox, and Deferiprone.

In other embodiments the antimicrobial agent used may be, Benzalkonium chloride (BAC), polyhexanide (PHMB, Polyhexamethylene Biguanide), chlorhexidine, EDTA, purite (stabilized oxychloro complex (SOC)), polyquad (polyquaternium-1), or SofZia. The formulation may also be packaged in preservative-free eye drop system which avoids the need for preservatives and prevents bacterial contamination. Such examples include Novelia, ABAK, COMOD.

In embodiments, the excipient is at least one of hydroxypropyl methyl cellulose, polyvinylpyrrolidone, ethanol, or dimethyl sulfoxide at a concentration of about 0.05% (w/v) to about 0.9% (w/v) or is about 0.05% (v/v) to about 1% (v/v).

In further embodiments, excipients may include, hydroxypropyl methylcellulose (HPMC), carbomer (carbopol-polyacrylic acid derivatives), sodium hyaluronate (hyaluronic acid), polyvinyl alcohol (PVA), polyethylene glycol (PEG) or propylene glycol (PPG), carboxymethylcellulose sodium-CMC, xanthan gum or combinations thereof. The excipient may be present in the ophthalmic formulation at about 0.05-1.0 (w/v).

Also disclosed herein are methods of preventing, treating and/or reducing a risk of corneal endothelial dysfunction in a subject. The methods comprise administering to the subject a formulation comprising a telomerase activator in an amount effective to prevent, treat and/or reduce a risk of the corneal endothelial dysfunction. Although the embodiments described herein can be administered as a ophthalmic eye drop formulation, those of skill in the art will understand that the active ingredient in the formulation can be administered to the subject in various forms, including as an injection, an eye drop solution, or a topical ointment.

In certain embodiments, the methods disclosed herein are related to the prevention, or treatment of corneal endothelial dysfunction, including Fuchs' endothelial corneal dystrophy (FECD), corneal edema, bullous keratopathy (PBK), acute or chronic corneal endothelial cell (CEC) loss, corneal degeneration, corneal melting, or corneal ectasia, or any combination thereof. Endothelial decompensation, which manifests as corneal opacity, arises from a variety of primary and secondary causes. The most common primary etiology is Fuchs' endothelial corneal dystrophy (FECD). Other inherited conditions like posterior polymorphous corneal dystrophy (PPCD), congenital hereditary endothelial dystrophy (CHED), and Iridocorneal Endothelial (ICE) Syndrome also contribute to endothelial dysfunction. Secondary causes often involve surgical trauma, particularly from cataract extraction and intraocular lens (IOL) implantation, leading to pseudophakic bullous keratopathy (PBK)—the most frequent secondary cause. Chemical injuries from preservatives in ophthalmic medications or improper rinsing of surgical instruments can further damage endothelial cells. Trauma, whether blunt or penetrating, and infections such as herpes simplex endotheliitis also compromise endothelial function. Additionally, glaucoma and its treatments (e.g., medications with toxic preservatives, surgical interventions like trabeculectomy, or previous failed corneal grafts) exacerbate endothelial cell loss.

The method comprises administering to the subject a formulation comprising the telomerase activator cycloastragenol, as detailed above. In embodiments, the formulation comprises the telomerase activator at a concentration of about 0.3 μg/ml to about 370 μg/ml.

In embodiments, the method comprises administering to the subject the telomerase activator at a concentration of 0.5 μg/ml-350 μg/ml, 10 μg/ml-320 μg/ml, 20 μg/ml-290 μg/ml, 30 μg/ml-270, 40 μg/ml-260 μg/ml, 50 μg/ml-250 μg/ml, or 80 μg/ml to about 240 μg/ml or any range or value therebetween.

Also disclosed is an eye drop formulation for treating, preventing and/or reducing a risk of a corneal endothelial dysfunction, wherein the eye drop formulation comprises a telomerase activator in an amount of 80 μg/ml to about 240 μg/ml, an antimicrobial agent, a chelator, and an excipient. More particularly the eye drop formulations disclosed herein are directed to treating or preventing Fuchs' endothelial corneal dystrophy (FECD), corneal edema, bullous keratopathy (PBK), acute or chronic corneal endothelial cell (CEC) loss, corneal degeneration, corneal melting, or corneal ectasia, and endothelial dysfunctions and traumas disclosed in more detail above.

The following examples are given purposes of illustrating various embodiments of the invention and are not meant to limit the present invention in any fashion.

### EXAMPLE 1

### Preparation of Eye Drop Formulation

### I. Chemicals

1. Hydroxypropyl methyl cellulose (HPMC), Pharma Grade, H3785 SIGMA-ALDRICH
2. Benzalkonium Chloride, Pharma Grade, 12063 SIGMA-ALDRICH
3. EDTA Disodium Salt 2-hydrate, Pharma Grade, 141669 APPLICHEM
4. Dimethyl Sulfoxide (DMSO), Pharma Grade, 191954 APPLICHEM
5. 0.9% (w/v) Isotonic sodium chloride solution for infusion
6. Cycloastragenol (Active Compound), Bionorm Natural Products Co. Ltd.

### Devices

1. Esco Labculture® Class 2 Type 2a Biosafety Cabinet
2. ORION 5-STAR pH/ISE/Conductivity/DO Benchtop Multiparameter Meter
3. BROOKFIELD DV-E Viscometer

### Formulation Eye Drop Content

1. (Hydroxypropyl) methyl cellulose (HPMC): 0.125% (w/v), 1.25 mg/ml
2. Benzalkonium Chloride: 0.01% (w/v), 0.1 mg/ml
3. EDTA Disodium Salt 2-hydrate: 0.01% (w/v), 0.1 mg/ml
4. 0.9% Isotonic Sodium Chloride Solution for infusion
5. Active compound active dose range: Cycloastragenol, 3 to 500 ng/ml
   Preparation of the Hydroxypropyl methyl cellulose (HPMC) Solution

HPMC solution was made using the ‘hot-melt’ method. 20 ml of isotonicsodium chloride (0.9% w/v) is heated to 80° C. and added to 125 mg of HPMC. The mixture was allowed to stand for 1 hour at room temperature. 40 ml of isotonic saline was added to this mixture to obtain a viscous solution of HPMC.

### Preparation of Benzalkonium Chloride Solution

10 mg of Benzalkonium Chloride was dissolved in 10 ml saline solution and added to the HPMC solution.

### Preparation of EDTA Disodium Solution

10 mg of EDTA Disodium salt was dissolved in 10 ml saline solution and added to the HPMC/Benzalkonium Chloride solution.

Preparation of Formulation eye drop

A 1 mg/ml main stock solution of the active compound, cycloastragenol was prepared in DMSO. The stock solution was diluted with DMSO to obtain three intermediate stock solutions having cycloastragenol at 0.5 mg/ml, 0.1 mg/ml and 0.025 mg/ml. To make Formulation eye drops having cycloastragenol at 25, 100 or 500 ng/ml, 100 μL of the appropriate intermediate stock solution was mixed with the 80 ml of HPMC/Benzalkonium Chloride/EDTA disodium solution prepared above, and the volume made to 100 ml using isotonic sodium chloride solution (0.9% w/v). The pH of the resulting solution was adjusted between 7.2 and 7.4 using 0.1 N sodium hydroxide. The solution was aseptically filtered through 0.22 um filters in a laminar flow cabin and stored in sterile amber colored bottles.

### Preparation of Placebo Eye Drop

For preparing the placebo eye drops, the same procedure was followed as described above except that cycloastragenol was omitted to give eye drops having just the carrier, DMSO.

Formulation and placebo eye drops were preserved by using cold chain technology and stored in a special refrigerator at 2°-4° C.

### Viscosity Measurements

The viscosity of the eye drop solution was measured using a Brookfield DV-E viscometer (Table 1).

### II. Stability of the Formulation Eye Drop Formulation

The formulation was tested for stability by microbiological testing and pH and viscosity measurements.

TABLE 1
Viscosity measurements of Formulation eye drops

| Cycloastragenol | | Speed | |
|---|---|---|---|
| concentration in | Torque | (revolutions | Viscosity |
| formulation eye drops | (%) | per minute) | (mPas) |
| 500 ng/ml | 10.8 | 100 | 6.96 |
| 100 ng/ml | 10.2 | 100 | 7.18 |
| 25 ng/ml | 10.6 | 100 | 7.24 |
| 0 ng/ml (Placebo) | 10.8 | 100 | 6.82 |

### Microbial Testing

Group N—negative control
Group A—placebo
Group B—Active compound (cycloastragenol), 25 ng/ml
Group C—Active compound (cycloastragenol), 100 ng/ml
Group D—Active compound (cycloastragenol), 500 ng/ml

The five groups of drugs above were incubated in the stability chamber at +4° C., +24° C. and +40° C. for 0, 3 and 6 months. At the end of the incubations, each group was subjected to stability test, sterility test, viscosity change test and pH change test. In addition, 6 months stability test groups were chromatographed by HPLC to verify the absence of degraded cycloastragenol. Tables 2 and 3 show representative integration results and peak analysis for the HPLC chromatogram of cycloastragenol after 6 months.

TABLE 2
Integration results from the HPLC chromatogram

| | Retention | | | Relative | Relative | |
|---|---|---|---|---|---|---|
| | Time | Area | Height | Area | Height | |
| No. | (min) | (mAU*min) | (mAU) | (%) | (%) | Amount |
| 1 | 1 | 249.386 | 135.355 | 6.41 | 1.91 | n.a. |
| 2 | 2.023 | 22.711 | 255.215 | 0.58 | 3.59 | n.a. |
| 3 | 2.09 | 24.996 | 266.542 | 0.64 | 3.75 | n.a. |
| 4 | 2.377 | 0.188 | 3.329 | 0 | 0.05 | n.a. |
| 5 | 2.52 | 45.477 | 1287.375 | 1.17 | 18.12 | n.a. |
| 6 | 2.57 | 139.271 | 2034.883 | 3.58 | 28.64 | n.a. |
| 7 | 3.08 | 2562.213 | 2410.722 | 65.9 | 33.93 | n.a. |
| 8 | 3.41 | 0.199 | 4.962 | 0.01 | 0.07 | n.a. |
| 9 | 4.127 | 10.677 | 41.953 | 0.27 | 0.59 | n.a. |
| 10 | 6.893 | 29.114 | 26.836 | 0.75 | 0.38 | n.a. |
| 11 | 8.697 | 133.511 | 41.635 | 3.43 | 0.59 | n.a. |
| 12 | 11.987 | 294.308 | 154.01 | 7.57 | 2.17 | n.a. |
| 13 | 14.17 | 50.054 | 114.164 | 1.29 | 1.61 | n.a. |
| 14 | 14.557 | 144.334 | 109.463 | 3.71 | 1.54 | n.a. |
| 15 | 15.967 | 27.767 | 94.767 | 0.71 | 1.33 | n.a. |
| 16 | 16.983 | 146.45 | 78.325 | 3.77 | 1.1 | n.a. |
| 17 | 18.763 | 3.369 | 18.833 | 0.09 | 0.27 | n.a. |
| 18 | 18.84 | 2.417 | 16.157 | 0.06 | 0.23 | n.a. |
| 19 | 19.043 | 1.352 | 9.295 | 0.03 | 0.13 | n.a. |
| 20 | 19.7 | 0.383 | 1.4 | 0.01 | 0.02 | n.a. |
| Total | — | 3888.177 | 7105.222 | 100 | 100 | — |

TABLE 3
Peak analysis of the HPLC chromatogram

| | Retention | Width | | | | |
|---|---|---|---|---|---|---|
| | Time | 50% | | Resolution | Asymmetry | Plates |
| No. | (min) | (min) | Type | (EP) | (EP) | (EP) |
| 1 | 1 | n.a. | BM | n.a. | n.a. | n.a. |
| 2 | 2.023 | n.a. | M | n.a. | n.a. | n.a. |
| 3 | 2.09 | n.a. | MB | n.a. | n.a. | n.a. |
| 4 | 2.377 | n.a. | BM | n.a. | n.a. | n.a. |
| 5 | 2.52 | n.a. | M | n.a. | n.a. | n.a. |
| 6 | 2.57 | n.a. | M | n.a. | n.a. | n.a. |
| 7 | 3.08 | 0.176 | M | n.a. | n.a. | 1692 |
| 8 | 3.41 | n.a. | Rd | n.a. | n.a. | n.a. |
| 9 | 4.127 | n.a. | Rd | n.a. | n.a. | n.a. |
| 10 | 6.893 | n.a. | Rd | n.a. | n.a. | n.a. |
| 11 | 8.697 | n.a. | Rd | n.a. | n.a. | n.a. |
| 12 | 11.987 | n.a. | M | n.a. | n.a. | n.a. |
| 13 | 14.17 | n.a. | M | n.a. | n.a. | n.a. |
| 14 | 14.557 | n.a. | M | n.a. | n.a. | n.a. |
| 15 | 15.967 | n.a. | M | n.a. | n.a. | n.a. |
| 16 | 16.983 | n.a. | M | n.a. | n.a. | n.a. |
| 17 | 18.763 | n.a. | M | n.a. | n.a. | n.a. |
| 18 | 18.84 | n.a. | M | n.a. | n.a. | n.a. |
| 19 | 19.043 | n.a. | MB | n.a. | n.a. | n.a. |
| 20 | 19.7 | 0.292 | BMB | n.a. | 1 | 25188 |

### Sterility Tests

Sterility tests were performed as described (1, 2). Sterility test results showed that there was no microbial growth in the 6 months stability test group at +4° C. and it was safe for use. Thus, the stability test results indicate that shelf life of the eye drop formulation was found to be 6 months at +4° C.

TABLE 4
Stability assessments from viscosity
and pH of formulation eye drops

| | Cycloastragenol | | | Viscosity | | |
|---|---|---|---|---|---|---|
| Time, | concentration | | | | Speed | |
| temper- | in formulation | | | Torque | (revolutions | Viscosity |
| ature | eye drops | | pH | (%) | per minute) | (mPas) |
| 0 days | 500 | ng/ml | 7.38 | 12.1 | 100 | 7.26 |
| | 100 | ng/ml | 7.35 | 12.4 | 100 | 7.44 |
| | 25 | ng/ml | 7.35 | 12.3 | 100 | 7.38 |
| | 0 ng/ml | | 7.34 | 12.2 | 100 | 7.32 |
| | (Placebo) | | | | | |
| 30 days, | 500 | ng/ml | 6.65 | 11.5 | 100 | 6.90 |
| 4° C. | 100 | ng/ml | 6.71 | 11.6 | 100 | 6.96 |
| | 25 | ng/ml | 6.71 | 11.2 | 100 | 6.72 |
| | 0 ng/mL | | 6.64 | 11.1 | 100 | 6.66 |
| | (Placebo) | | | | | |
| 30 days, | 500 | ng/ml | 6.60 | 11.4 | 100 | 6.84 |
| 25° C. | 100 | ng/ml | 6.77 | 11.2 | 100 | 6.72 |
| | 25 | ng/ml | 6.90 | 11.3 | 100 | 6.78 |
| | 0 ng/mL | | 6.68 | 11.0 | 100 | 6.60 |
| | (Placebo) | | | | | |
| 30 days, | 500 | ng/ml | 6.64 | 11.6 | 100 | 6.96 |
| 40° C. | 100 | ng/ml | 6.76 | 11.1 | 100 | 6.66 |
| | 25 | ng/ml | 6.72 | 11.4 | 100 | 6.84 |

| | | | | |
|---|---|---|---|---|
| 0 ng/ml | 7.24 | 10.8 | 100 | 6.48 |
| (Placebo) | | | | |

### Viscosity and pH Measurements

The formulations were kept in a time and temperature-controlled stability cabinet at +25° C. (Room Temperature), +40° C. and +4° C. for 0 and 30 days followed by viscosity and pH measurements. Table 4 shows the results of pH and viscosity measurements after exposing the formulation for 30 days at 4° C., 25° C. and 40° C.

A decrease in the pH of the eye drops to 6.6 was observed after 30 days. 1O On the other hand, the decrease in viscosity observed is negligible and would not be expected to affect activity.

### EXAMPLE 2

### Cell Culture Studies

All experiments were designed and carried out in a randomized, double blind manner using placebos.

### Cytotoxicity Analysis

Corneal endothelial cell HCEC-B4G12 was used to determine in vitro effects of Formulation eye drop formulation on cell viability. Cytotoxicity was analyzed using the 2,5-Diphenyltetrazolium Bromide (MTT) assay at cycloastragenol concentrations ranging from 3.1 ng/ml and 100 μg/ml.

### Telomerase Activity

Effect of Formulation eye drop formulation on telomerase activity was evaluated in cornea endothelial cell HCEC-B4G12 using commercially available TeloTAGGG™ Telomerase PCR ELISA assay. The assay was performed 30 h after incubating the cells with the formulation containing 3.1 ng/ml to 100 μg/ml cycloastragenol.

### Results

### Cytotoxicity Analysis

The corneal cell line HCEC-B4G12 (DSMZ, ACC 647) was used to examine the dose and time dependent in vitro effect of the Formulation eye drop on cell viability of corneal endothelial cells as well as its cytotoxic activity. Subculture, incubation, harvest and storage transactions were made in compliance with the protocol that DSMZ suggests for this cell line. Formulation eye drop containing cycloastragenol was prepared fresh for each treatment as described above. The cells were treated with the formulation to get final cycloastragenol concentrations between 3.1 ng/ml and 100 μg/ml. After 48 hours, the effect of the medicine on cell viability and/or cytotoxic activity was determined using the 3-(4,5-Dimethylthiazol-2-yl)-2,5-Diphenyltetrazolium Bromide (MTT) test. Data are mean of three independent experiments each repeated thrice.

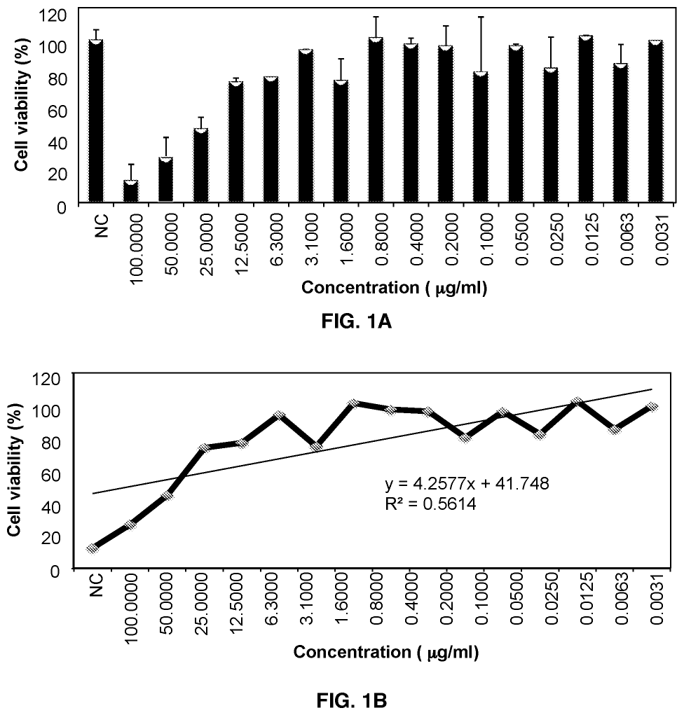

In general, it was observed that cell viability decreased as the cycloastragenol concentration was increased. Assuming the cell viability as 100% in the negative control (1% DMSO) and when the other values are normalized based on this value, cell viability values compared to the concentrations determined are shown in Table 5. IC₅₀ value was calculated as 40.2 μg/ml (Table 5; FIG. 1A-1C).

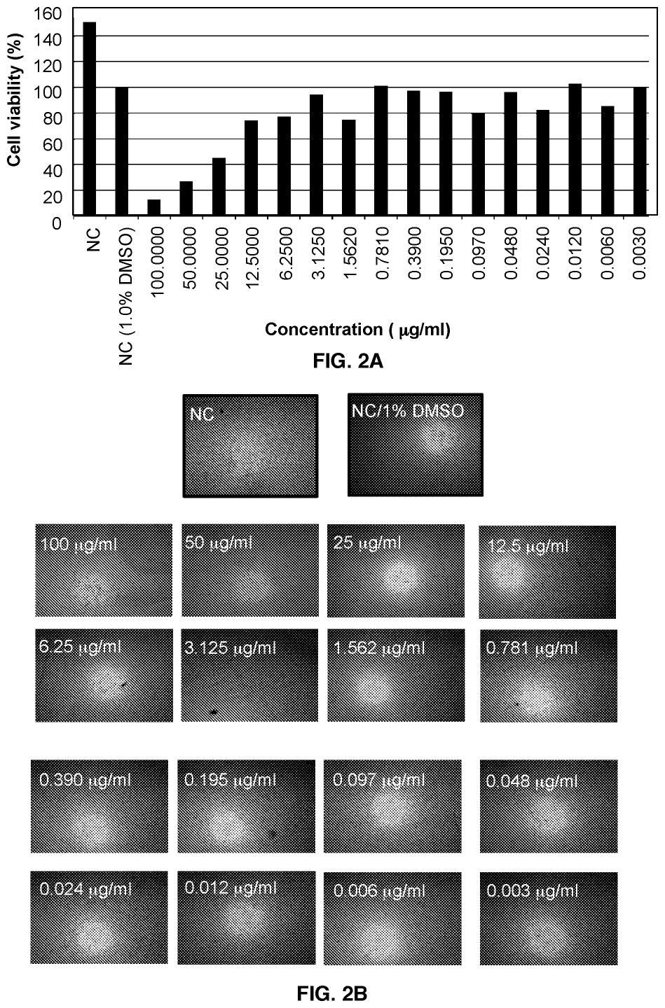

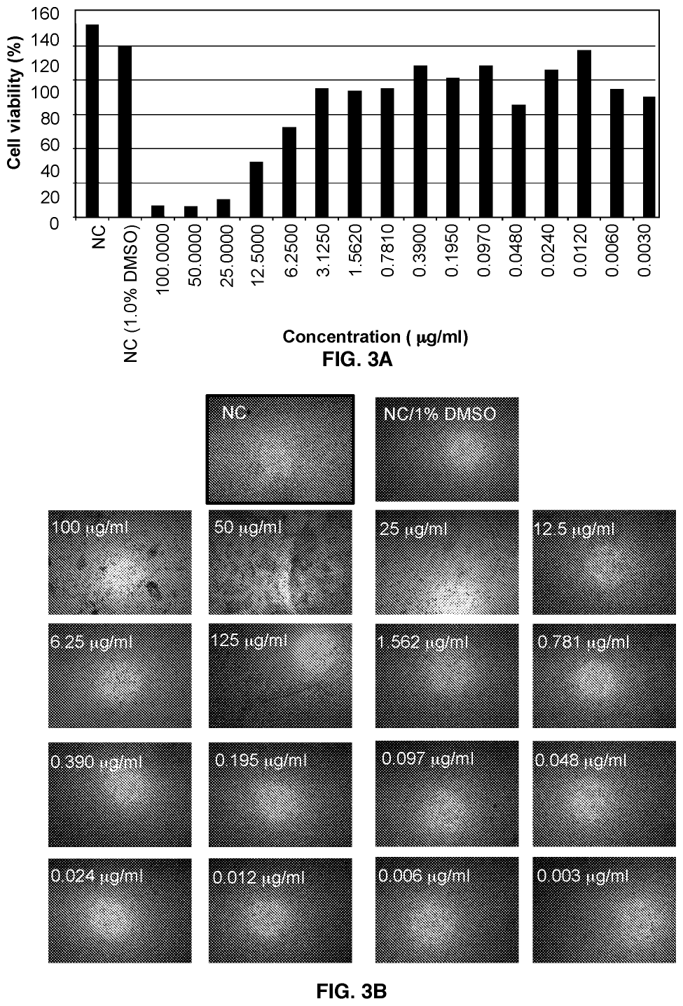

FIGS. 2A and 3A show cell viability data for corneal endothelial cells incubated with Formulation eye drops in 1% and 0.2% DMSO respectively, and FIGS. 2B and 3B show representative photomicrographs of the control and treated corneal endothelial cells.

TABLE 5
Cell Viability Values

| Concentration (μg/mL) | % Cell Viability ± Std.Dev. |
|---|---|
| 0* | 100 |
| 100 | 12.79 ± 6.30 |
| 50 | 27.02 ± 10.72 |
| 25 | 44.99 ± 13.09 |
| 12.5 | 74.06 ± 7.58 |
| 6.3 | 77.11 ± 2.62 |
| 3.1 | 94.18 ± 0.02 |
| 1.6 | 74.95 ± 0.07 |
| 0.8 | 101.41 ± 13.42 |
| 0.4 | 97.49 ± 12.87 |
| 0.2 | 96.4 ± 3.66 |
| 0.1 | 80.38 ± 12.22 |
| 0.05 | 96.25 ± 33.80 |
| 0.025 | 82.41 ± 1.30 |
| 0.0125 | 102.63 ± 19.31 |
| 0.00625 | 85.39 ± 0.23 |
| 0.00313 | 99.57 ± 11.90 |

Cells were treated with the active compound (Formulation) in an eye drop. Data shown were obtained after 48 hours. Results are mean of 3 independent experiments, each repeated thrice.

### TeloTAGGG™ Telomerase Activity

Corneal cell line HCEC-B4G12 (Deutsche Sammlung von Mikroorganismen und Zellkulturen (DSMZ) ACC-647) has been used in the study of researching the dose dependent in vitro effect of the Formulation eye drop on telomerase enzyme activity. Subculture, incubation, harvest and storage transactions were made in compliance with the protocol that DSMZ suggests for this cell line.

The Formulation eye drop containing cycloastragenol was prepared fresh for each treatment as described above. Cells were treated with Formulation eye drop formulation to get final cycloastragenol concentrations from 3.7 ng/ml to 10 μg/mL. The effect of Formulation on telomerase enzyme activity was determined using a TeloTAGGG™ Telomerase PCR ELISA assay (Roche, CAS Number 11854666910,). TeloTAGGG™ Telomerase PCR ELISA assay was performed as described by manufacturer (Sigma-Aldrich).

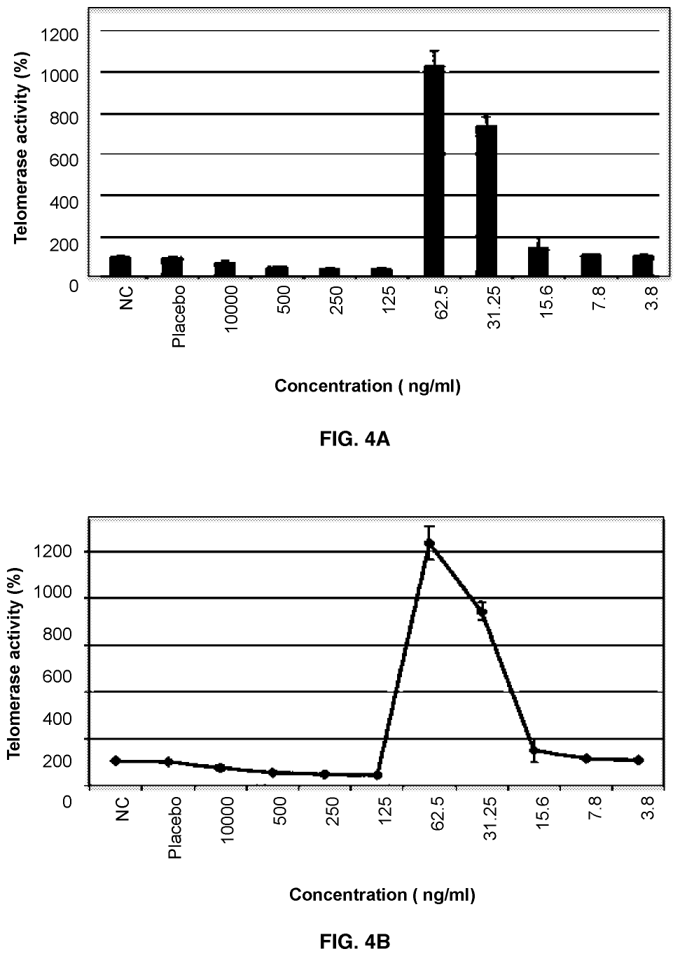

High Formulation concentrations in the range of 125-1000 ng/ml were found to be lower than negative control and lower Formulation concentrations in the range of 3.8-62.5 ng/ml were found to have higher telomerase activity than negative control. The highest activity of telomerase was obtained at 62.5 ng/ml Formulation. The telomerase activity obtained at this concentration was 10.35 times higher than the negative control (Table 6, FIGS. 4A and 4B). Cycloastragenol showed very high telomerase enzyme activity of corneal cells (HCEC-B4G12) in the concentration range of 62.5-31.25 ng/ml (1034.92-741.35%, respectively). This activity is approximately 7-10 times higher than the negative control. The activity decreases dramatically at the application concentrations below and above these concentrations (Table 4, FIGS. 4A and 4B). Assuming the telomerase enzyme activity is 100% in the negative control containing 1% DMSO (placebo) and when the other values are normalized based on this value, telomerase enzyme activity values compared to the concentrations are determined as shown in Table 6 and FIGS. 4A and 4B.

### EXAMPLE 3

### Animal Studies

All experiments were designed and carried out in a randomized, double blind manner using placebos. Rabbits were housed for one week prior to the study, fed on standard laboratory food and allowed free access to water in a room at standard temperature and humidity conditions in a 12-h light/12-h dark cycle.

TABLE 6
Telomerase Enzyme Activity

| Treatment | % Telomerase Activity ± SD |
|---|---|
| NC | 103.8 ± 4.7 |
| Placebo | 100.00 |
| Concentration | |
| (ng/ML) | |
| 10000 | 74.0 ± 10.45 |
| 500 | 54.38 ± 2.78 |
| 250 | 46.28 ± 2.02 |
| 125 | 43.14 ± 0.24 |
| 62.5 | 1034.92 ± 69.41 |
| 31.25 | 741.35 ± 38.49 |
| 15.6 | 148.94 ± 49.54 |
| 7.8 | 114.42 ± 2.79 |
| 3.8 | 108 ± 5.27 |

The corneal cell line HCEC-B4G12 was treated with the active compound (Formulation) in an eye drop. At a concentration range from 3.8 to 10000 ng/ml for 30 hours. Data were result of 3 independent experiments, each repeated thrice.

### Animal Model and Surgical Procedure

Rabbits were randomly divided and distributed into five groups:
Group N—negative control
Group A—placebo
Group B—Active compound (cycloastragenol), 25 ng/ml
Group C—Active compound (cycloastragenol), 100 ng/ml
Group D—Active compound (cycloastragenol), 500 ng/ml
Four rabbits (eight eyes) were used per group. This design provided a statistical significance p 0.001.

### Counting Corneal Cell Number

Each rabbit was treated with 0.1 ml of eye drop formulation twice a day for six months. The formulation was prepared in 15-day volumes. Any excess was discarded, and a fresh batch prepared for the next 15-days. Effects of the eye drop on corneal endothelium as well as cell numbers and shapes were observed by means of specular microscopy (NIDEK-CEM 530, Japan) at the end of months 0, 1, 3 and 6. Rabbits were euthanatized by an intravenous injection of Tanax (0.3 ml/kg). Vitreous (0.2 ml) was aspirated with a 25-gauge needle attached to a 5-ml disposable syringe, driven at 3.0 mm from the limbus, and guided toward the center of the eyeball, taking care to avoid bleeding when the needle was introduced (vitreous samples were stored at −80° C. until the analysis). Eye tissue was fixed with 4% formaldehyde and embedded in paraffin for histopathological assessment. The individual values for three replicates were determined and the mean values were reported.

### Statistical Analysis and Methods

The statistical significance of the differences between the concentrations of active compound following administration of the drops and the corresponding controls was tested at each time point by a one-way analysis of variance (ANOVA) with the pairwise multiple comparison procedures (Student-Newman-Keuls Method) for multiple comparison (SigmaStat program; Jandel Scientific, Version 1.0) and expressed as means±standard error (SEM) of three independent experiments. All analyses were carried out using GraphPad Prism 5.0 (GraphPad Software, San Diego, CA, USA). Differences were considered to be significant at a level of P: : : : 0.001.

### Histopathology

Eye tissue was fixed with 4% formaldehyde and embedded in paraffin for the histopathological assessment and processed according to routine light microscope tissue processing methods. 5 μm tissue sections stained with Hematoxylin-Eosin (H&E) were examined and photographed by Leica image analyzing system (Leica, Germany). All specimens were evaluated individually by three histologists who were blinded to the drug type and the treatment times. Histopathology was also performed to evaluate the corneal opacity, neovascularization, inflammatory cell density, vessel size and edema. Moreover, lens structure, scleral, choroidal and macular changes were analyzed by histopathology.

### Telomere Length Analysis

Samples were received in paraffin. Different methods were used in order to obtain the best results during the dewaxing process. After dewaxing, the tissue samples were processed according to Life Length's cell disaggregation protocol to generate single-cell suspensions appropriate for the analysis. This was followed with a filter step to eliminate collagen residues in the preparation.

### Results

### Corneal Endothelial Cells Counting

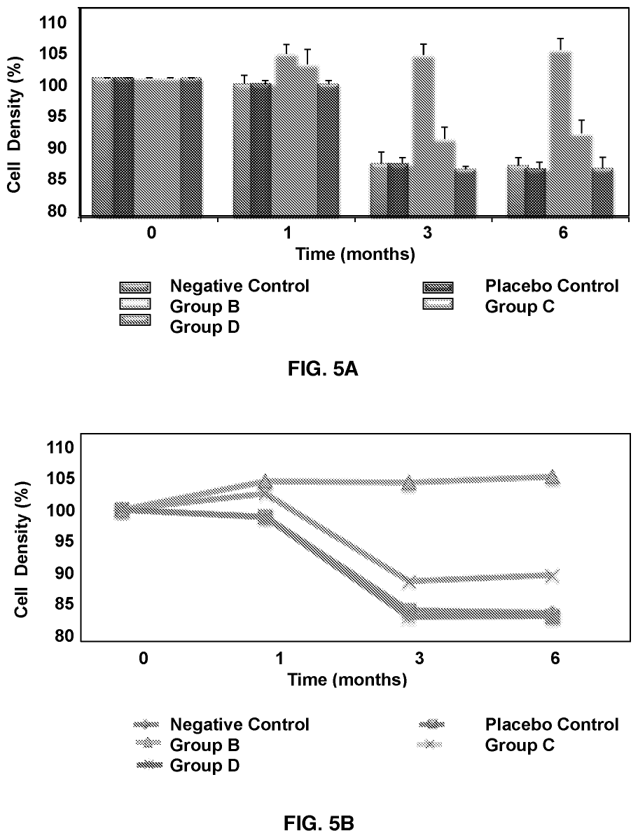

Cornea layers from the eyes of untreated (negative control, Group N), carrier treated (placebo, Group A) and cycloastragenol-treated (Groups B, C and D) rabbits were analyzed by specular microscopy at the end of 0, 1, 3 and 6 months. Time dependent changes in the density of corneal endothelial cells (CD parameter) were compared and these are presented in Tables 5-8 and in FIGS. 5-7. The data shows that relative change in corneal endothelial cell density in the negative control group was 99.12 (±1.22) at the end of month 1, 87.74 (±1.61) at the end of month 3 and 87.45 (±1.14) at the end of month 6 (normalized to month 0), which corresponds to a 1%, 12% and 13% reduction in cell density respectively. At the end of month 6, it was observed that the endothelial cells in this group degenerated by 13% as a result of apoptosis and/or autophagy (y=−4.9028x+105.84; R2=0.8372) (Table 7, FIGS. 5A and 5B). In the placebo group, corneal endothelial cell density was found to be 99.17 (±0.43) at the end of month 1, 87.72 (±0.85) at the end of month 3 and 87.00 (±0.92) at the end of month 6, similar to the negative control group (y=−5.0455x +106.09; R2=0.8479) (Table 7, 10; FIGS. 5A and 5B). Thus, time dependent changes in the density (CD parameter) of endothelial cells in cornea layers of the eyes from the negative control and placebo experimental group have very similar values and they change parallel to each other suggesting that the carrier does not have any negative effect on cell viability.

TABLE 7
Relative changes to cell density following Formulation treatment

| | CD ± SEM | | | |
|---|---|---|---|---|
| Time (months) | 0 | Time (months) | 0 | Time (months) |
| Group N | 100.00 + | Group N | 100.00 ± | Group N |
| (Negative | 0.00 | (Negative | 0.00 | (Negative |
| Control) | | Control) | | Control) |
| Group A | 100.00 ± | Group A | 100.00 ± | Group A |
| (Placebo) | 0.00 | (Placebo) | 0.00 | (Placebo) |
| Group B | 100.00 ± | Group B | 100.00 ± | Group B |
| | 0.00 | | 0.00 | |
| Group C | 100.00 ± | Group C | 100.00 ± | Group C |
| | 0.00 | | 0.00 | |
| Group D | 100.00 ± | Group D | 100.00 ± | Group D |
| | 0.00 | | 0.00 | |

Relative changes to cell density (CD parameter) of cornea layer endothelial cells in negative control (no treatment), placebo (carrier alone) and Formulation treatment groups B, C and D containing cycloastragenol at 25, 100 and 500 ng/ml respectively. Results are normalized to month 0 and shown at the end of months 0, 1, 3 and 6.

For the treatment group B (25 ng/ml), the corneal endothelial cell density was 103.44 (±1.34) for month 1, 103.29 (±1.53) for month 3 and 104.00 (±1.61) for month 6, which corresponds to a 3%, 3% and 4% change in cell density respectively. Thus, apoptosis and/or autophagy (natural cell death) in group B slows down and/or stops as evidenced by an increase in density by 4% (y=1.848x+99.72; R2=0.7109), (Table 6, FIGS. 5A and 5B).

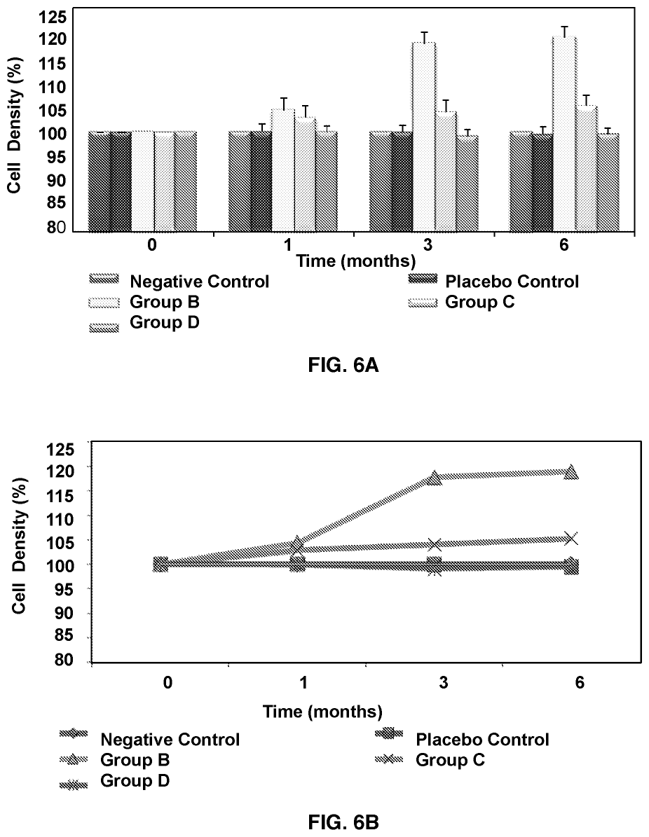

Comparison of relative changes to density of corneal layer endothelial cells in the negative control group and treatment group B shows a higher cell density for group B at about 4% in month 1, 18% in month 3 and 19% in month 6 (y=7,0134x +92,716; R2=0,9082), (Table 8, FIGS. 6A and 6B). At the end of the month 6, endothelial cells from group B was 19% higher in density compared to the negative control group. Consequently, compared to the negative control group, the effect of 25 ng/ml cycloastragenol on slowing down and stopping apoptosis and/or autophagy as a consequence of natural cell aging was determined to be 100%, which corresponds to a 0% degeneration rate (Table 8, FIGS. 6A and 6B).

TABLE 8
Relative changes to cell density following Formulation treatment

CD ± SEM
Time (months)

| | 0 | 1 | 3 | 6 |
|---|---|---|---|---|
| Group N | 100.00 ± | 100.00 ± | 100.00 ± | 100.00 ± |
| (Negative | 0.00 | 0.00 | 0.00 | 0.00 |
| Control) | | | | |
| Group A | 100.00 ± | 100.05 ± | 99.97 ± | 99.49 ± |
| (Placebo) | 0.00 | 1.52 | 1.33 | 1.61 |
| Group B | 100.00 ± | 104.36 ± | 117.72 ± | 118.93 ± |
| | 0.00 | 1.11 | 1.38 | 1.17 |
| Group C | 100.00 ± | 102.86 ± | 104.02 ± | 105.24 ± |
| | 0.00 | 2.49 | 2.35 | 2.19 |
| Group D | 100.00 ± | 99.99 ± | 99.09 ± | 99.54 ± |
| | 0.00 | 1.16 | 1.47 | 1.25 |

Relative changes to cell density (CD parameter) of cornea layer endothelial cells in negative control (no treatment), placebo (carrier alone) and Formulation treatment groups B, C and D containing cycloastragenol at 25, 100 and 500 ng/ml respectively. Results are normalized to Group N and shown at the end of months 0, 1, 3 and 6.

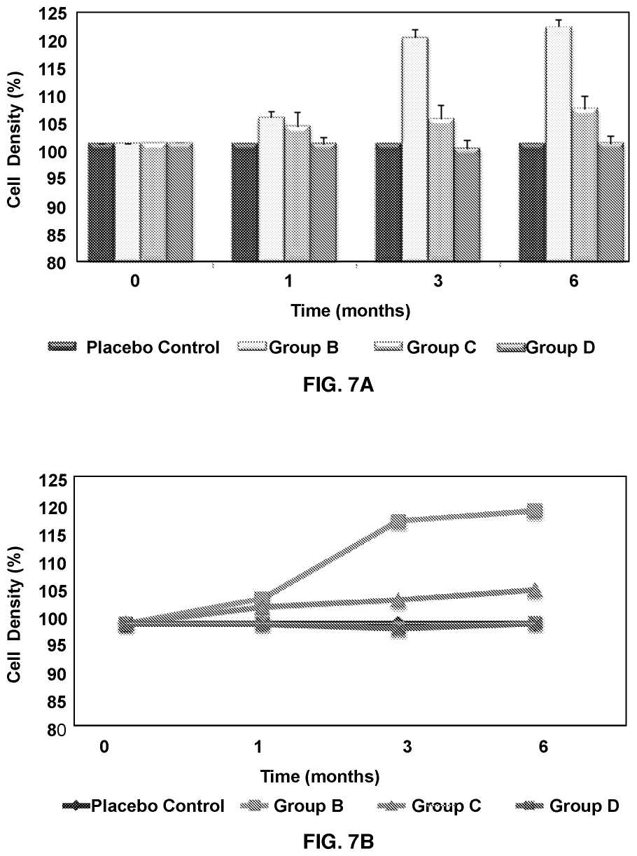

Comparison of relative changes to density of corneal layer endothelial cells in the placebo group and treatment group B shows a higher cell density for group B at about 4% in month 1, 18% in month 3 and 20% in month 6 (y=7,2068x+92,382; R2=0.9179) (Table 9, FIGS. 7A and 7B). At the end of the month 6, endothelial cells from group B was 20% higher in density compared to the placebo group. Consequently, compared to the placebo group, the effect of 25 ng/ml cycloastragenol on slowing down and stopping apoptosis and/or autophagy as a consequence of natural cell ageing was determined to be 100%, which corresponds to a 0% degeneration rate (Table 9, FIGS. 7A and 7B). Table 10 summarizes the results of cell density for the various treatment groups.

A significance analysis comparison between treatment groups using T-Student analysis is shown. Key: p>0.1 not significant; * p<0.01; ** p<0.0001.

### Histopathological Analysis

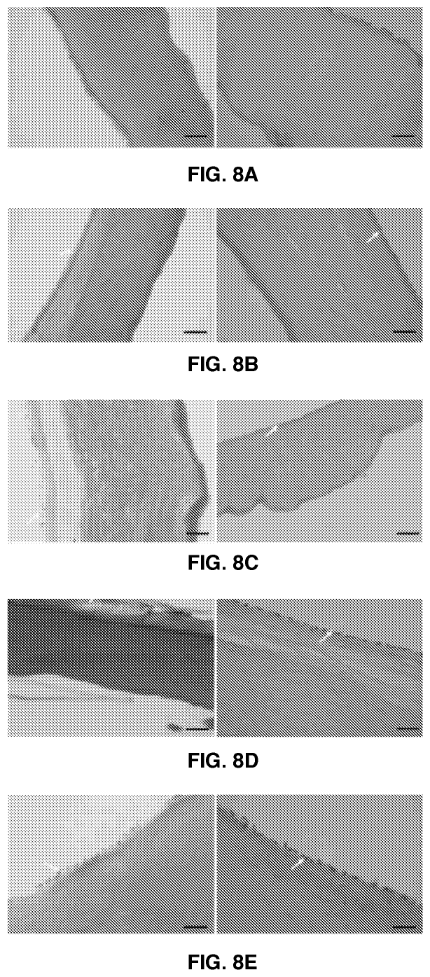

Group N (Negative control): Corneal endothelium was observed to form a layer of single-layer, hexagonal cells located on the descement membrane. Corneal endothelial cells were found to have a regular hexagonal alignment. Cell configurations in this group were found to be compatible with maternal corneal endothelium. No loss was found in the number of cells (FIG. 8A).

TABLE 9
Relative changes to cell density following Formulation treatment

CD ± SEM

Time (months)

| | 0 | 1 | 3 | 6 |
|---|---|---|---|---|
| Group A | 100.00 ± | 100.00 ± | 100.00 ± | 100.00 ± |
| (Placebo) | 0.00 | 0.00 | 0.00 | 0.00 |
| Group B | 100.00 ± | 104.31 ± | 117.75 ± | 119.54 ± |
| | 0.00 | 1.11 | 1.38 | 1.17 |
| Group C | 100.00 ± | 102.81 ± | 104.05 ± | 105.79 ± |
| | 0.00 | 2.49 | 2.35 | 2.19 |
| Group D | 100.00 ± | 99.94 ± | 99.12 ± | 100.05 ± |
| | 0.00 | 1.16 | 1.47 | 1.25 |

Relative changes to cell density (CD parameter) of cornea layer endothelial cells in negative control (no treatment), placebo (carrier alone) and Formulation treatment groups B, C and D containing cycloastragenol at 25, 100 and 500 ng/ml respectively. Results are shown at the end of months 0, 1, 3 and 6.

TABLE 10
Cell Density (CD) of cornea layer endothelial
cells in eyes from treated animals

CD ± SEM

| Time | 0 | 1 | 3 | 6 |
|---|---|---|---|---|
| Group N | 2715.83 ± | 2681.75 ± | 2401.00 ± | 2340.80 ± |
| (Negative) | 235.02 | 143.43 | 87.97 | 57.68 |
| Group A | 2903.0 ± | 2870.5 ± | 2438.50 ± | 2319.75 ± |
| (Placebo | 170.75 | 60.84 | 104.55 | 104.05 |
| Control) | | | | |
| Group B | 2531.00 ± | 2612.38 ± | 2604.50 ± | 2545.88 ± |
| | 211.52 | 129.09 | 78.17** | 45.42* |
| Group C | 2689.88 ± | 2741.13 ± | 2453.88 ± | 2472.38 ± |
| | 91.28 | 141.29 | 125.12 | 37.90** |
| Group D | 2875.13 ± | 2835.38 ± | 2498.50 ± | 2488.88 ± |
| | 251.01 | 177.28 | 331.49 | 241.53 |

*p ≤ 0.001,
**p ≤ 0.01.

Group A (Placebo): Corneal endothelium was found to consist of single-layer cells placed on the descement membrane. Where there are cell losses in place, histopathological degenerative effects were observed in terms of cell nuclei and their shape. It is concluded that this is compatible with the surgical intervention. It was observed that the nuclei of some cells increased in size and lost their natural properties (FIG. 8B).

Group B: Corneal endothelium consisted of single-layer cells placed on the descement membrane. Compared to group A, cell losses were observed in very few areas. Cell nuclei were found to be in proper configuration compared to Group A Group D and Group C compared the number of cell losses in the group where the cell configuration and number of the group was found to be the closest to Group NC (Figure SC).

Group C: Corneal endothelium was also seen in this group that formed a layer of single-layer; hexagonal cells located on the descement membrane. Group B and Group D compared with the number of cell losses seen in this group in the group where the configuration and number of cells were found to be close to Group NC (FIG. 8D).

Group D: The corneal endothelium was found to form a single-layer, hexagonal close-layered layer on the descement membrane. Although cell configurations were found to be compatible with mature corneal endothelium in the samples belonging to this group, compared to Group Band Group C, the more hypertrophic and place cell losses of the cells were found to be different from these two groups (Figure SE).

### Measurement of Telomerase Length in Corneal Endothelial Cells From Treated Animals

Six-month-old male New Zealand rabbits were treated (100 μI/eye, 6 months) with negative control (Group N), placebo (Group A) or the formulation of the present invention containing 25, 100, 500 ng/ml cycloastragenol (Groups B, C, D).

TABLE 11
Telomere length results from TAT analysis

| | | | 2am | | |
|---|---|---|---|---|---|
| | | Median | Percentile | | |
| | | Length | Length | | |
| | | (Base | (Base | Telomeres | |
| Formulation | Group | Pairs) | Pairs) | <3 Kbp (%) | CV % |
| Negative | GROUP | 10,310 | 6,123 | 5.2 | 0.7 |
| Control | N | | | | |
| O ng/ml | GROUP | 10,274 | 6,362 | 5.4 | 0.6 |
| (Placebo) | A | | | | |
| 25 ng/ml | GROUP | 11,431 | 6,968 | 3.7 | 1.1 |
| | B | | | | |
| 100 ng/ml | GROUP | 11,543 | 7,374 | 3.1 | 1.2 |
| | C | | | | |
| 500 ng/ml | GROUP | 11,552 | 7,113 | 3.6 | 2 |
| | D | | | | |

Median telomere length and 20th percentile median telomere length is shown for each treatment group. The percentage of short telomeres (<3 Kbp) are also shown.
Data are mean from five experiments.

Samples of the eye tissue were fixed and received in paraffin. After dewaxing, the tissue samples were processed according to Life Length's cell disaggregation protocol comprising mechanical and enzymatic steps to generate single-cell suspensions appropriate for the analysis. The sample is filtered to remove collagen residues. Telomere lengths were assessed using Life Length's.

TABLE 12
T-Student analysis of telomere length

| | Median | | 20 m | | Telomeres <3 | |
|---|---|---|---|---|---|---|
| | Length | | PercentileLength | | Kbpp | |
| Comparison | p Value | Significance | p Value | Significance | Value | Significance |
| Group A | 0.00005 | **** | 0.03 | * | 0.12 | not |
| vs | | | | | | significant |
| Group B | | | | | | |
| Group A | 0.0003 | *** | 0.002 | ** | 0.007 | ** |
| vs | | | | | | |
| Group C | | | | | | |
| Group A | 0.0001 | *** | 0.012 | * | 0.02 | * |
| vs | | | | | | |
| Group D | | | | | | |
| Group A | 1 | not | 0.5 | not | 0.6 | not |
| vs | | significant | | significant | | significant |
| Group N | | | | | | |
| Group B | 0.7 | not | 0.1 | not | 0.3 | not |
| vs | | significant | | significant | | significant |
| Group C | | | | | | |
| Group B | 0.3 | not | 0.6 | not | 0.7 | not |
| vs | | significant | | significant | | significant |
| Group D | | | | | | |
| Group B | 0.002 | ** | 0.02 | * | 0.08 | not |
| vs | | | | | | significant |
| Group N | | | | | | |
| Group C | 0.8 | not | 0.3 | not | 0.2 | not |
| vs | | significant | | significant | | significant |
| Group D | | | | | | |
| Group C | 0.002 | ** | 0.003 | ** | 0.02 | * |
| vs | | | | | | |
| Group N | | | | | | |
| Group D | 0.003 | ** | 0.011 | * | 0.04 | * |
| vs | | | | | | |
| Group N | | | | | | |

A significance analysis comparison between treatment groups using T-Student analysis isshown.
Key: p > 0.1 not significant;
* p < 0.05;
** p < 0.01;
*** p < 0.001;
**** p < 0.0001.ian

### TAT Analysis Protocol

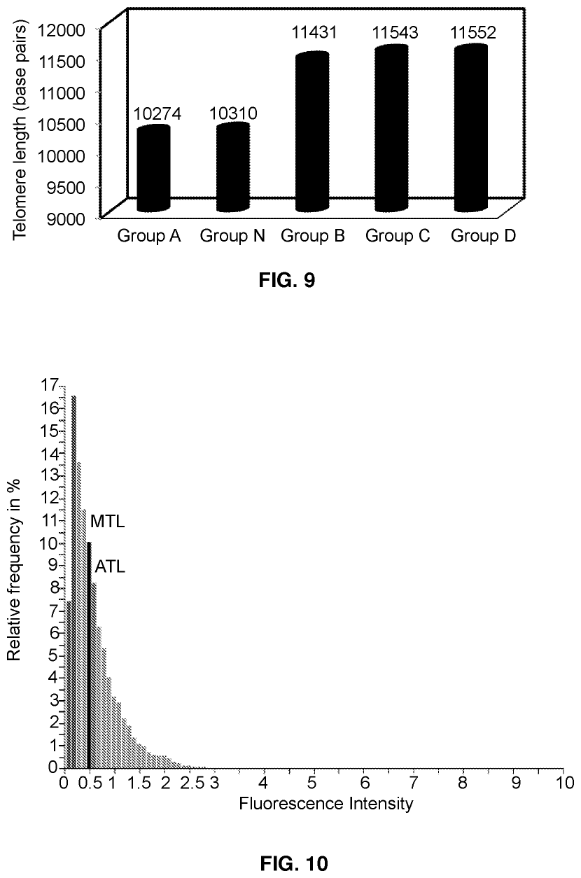

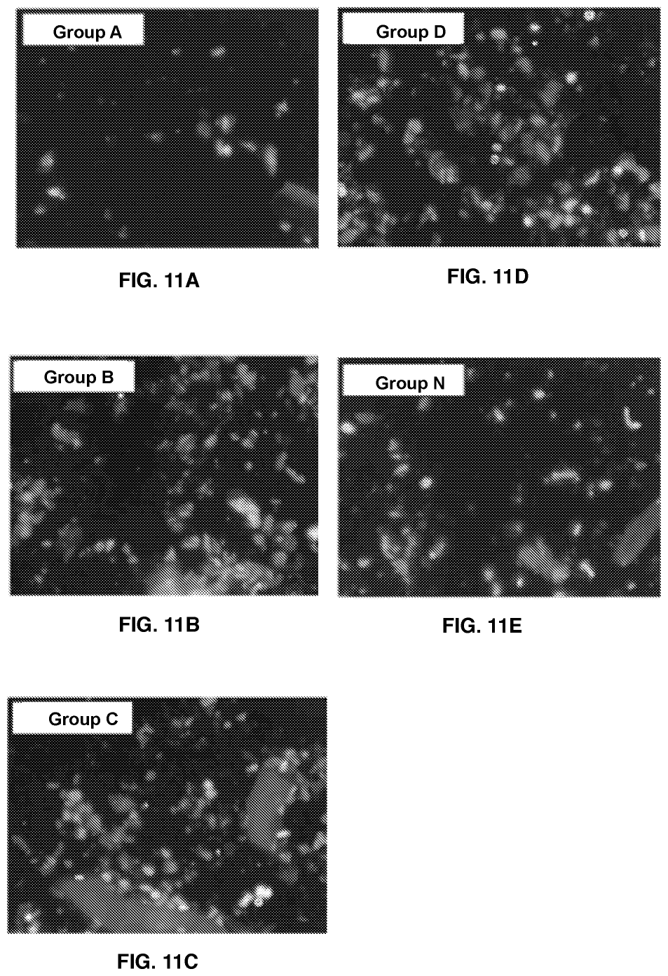

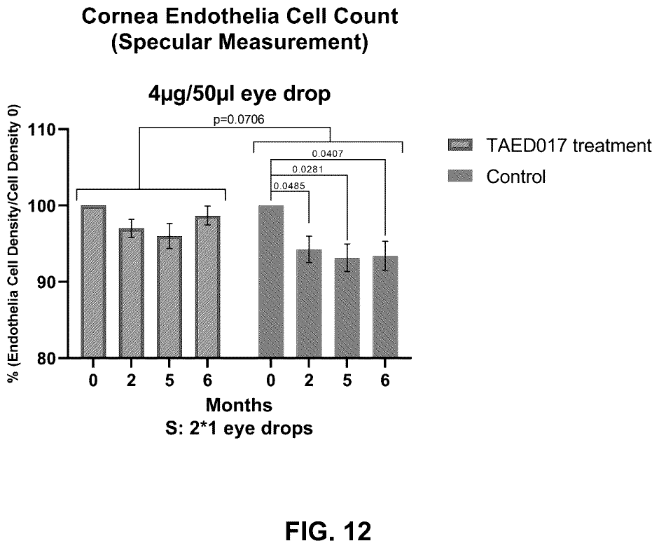

Table 11 shows the median telomere length and 20th percentile median telomere length (both in base pairs—bp) for each sample, as well as the percentage of short telomeres defined as the percentage of the telomeres with a length below 3 Kbp (<3 Kbp). All measurements were performed in quintuplicate. The T-Student analysis for significance is summarized in Table 12. FIG. 9 is a graphical presentation of the telomere length analysis. FIG. 10 shows a representative histogram showing distribution of telomere lengths. FIGS. 11A-11E show representative images from the treatment groups used for telomere length analysis in corneal endothelial cells.

### EXAMPLE 4

A Cycloastragenol master stock solution is prepared. 90 μl of absolute alcohol is added to 45 mg Cycloastragenol and vortexed for 10 minutes. Then 20 ml with 0.9% NaCl (FTS) is prepared, vortexed for 5 minutes and homogenized in the sonicator for 15 minutes to prepare the suspension. This Master stock solution has a cycloastragenol concentration of 2250 μg/ml.

Additional component preparation: 18.7 mg EDTA were added to 10 ml of FTS. 18.7 mg Benzalkonium were added to 10 ml FTS. 233.75 mg HPMC is mixed in 100 ml FTS on a magnetic stirrer at 45 degrees C. until dissolved.

Preparation of 80 μg/ml (4 μg/50 μl) Eye Drop Formulation

The following components were prepared and combined into a formulation for preparing an eye drop composition having 80 μg/ml of the active ingredient: 6.67 ml of the master stock Cycloastragenol solution, 10 ml EDTA, 10 ml Benzalkonium, 100 ml HPMC. By adding 60.83 ml of FTS, the total volume is adjusted to 187.5 ml and mixed in a magnetic stirrer. pH is measured and adjusted to 7.4 when necessary. It is passed through a 0.45 μm filter and filled into falcon tubes (15 ml) under the laminar cabin.

Preparation of 160 μg/ml (8 μg/50 μl) Eye Drop Formulation

The following components were prepared and combined into a formulation for preparing an eye drop composition having 160 μg/ml of the active ingredient: 13.37 ml of the master stock Cycloastragenol solution, 10 ml EDTA, 10 ml Benzalkonium, 100 ml HPMC. By adding 54.17 ml of FTS, the total volume is adjusted to 187.5 ml and mixed in a magnetic stirrer. pH is measured and adjusted to 7.4 when necessary. It is passed through a 0.45 μm filter and filled into falcon tubes (15 ml) under the laminar cabin.

Preparation of 240 μg/ml (12 μg/50 μl) Eye Drop Formulation

The following components were prepared and combined into a formulation for preparing an eye drop composition having 240 μg/ml of the active ingredient: 20 ml of the master stock Cycloastragenol solution, 10 ml EDTA, 10 ml Benzalkonium, 100 ml HPMC. By adding 47.5 ml of FTS, the total volume is adjusted to 187.5 ml and mixed in a magnetic stirrer. pH is measured and adjusted to 7.4 when necessary. It is passed through a 0.45 μm filter and filled into falcon tubes (15ml) under the laminar cabin.

### Safety and Efficacy of Cycloastragenol in Human Cornea Epithelia and Endothelia

A study was conducted with the aim to determine safe concentrations of cycloastragenol on human corneal epithelial and endothelial cells and to evaluate its effectiveness on telomerase activity at sub IC50 doses. With this study, the aim was to demonstrate for the first time the effectiveness of cycloastragenol as a telomerase activator of human corneal epithelial and endothelial cells.

In the morphologic evaluation, the cells at different time periods (1 h, 4 h, 24 h) were observed following different concentrations of drug administration. In the cornea endothelia, it was observed that cell-cell connections were disrupted and the cells became round in corneal endothelial cells at high concentrations e.g. 320 μM, 640 μM and 960 μM as early as 5 minutes. After 1 h and 4 h at these doses, 1-5 apoptotic cells and cytoplasmic vacuoles were observed in 20× microscope field. At doses of 40 μM and 80 μM, no significant changes were detected up to 24 hours. IC50 dose of TAED017 was calculated as 220 μM for cornea endothelial cells according to cell viability assays.

For cornea epithelial cells, degenerative changes were characterized by disruption of cell-cell junctions, lysis and round cell morphology at high doses such as 640 μM and 1280 μM doses, while 25-75% of the cells were completely lysed at 24 hours. However, concentrations of 160 μM and lower did not show morphological degenerative changes in epithelial cells and were shown to be safe. IC50 dose of TAED017 was calculated as 740 μM for cornea epithelial cells according to cell viability assays.

It was shown that telomerase activity of cornea endothelial and epithelial cells increased in direct proportion to the increasing dose of TAED017, starting from doses as low as 2 μM, 5 μM and 10 μM and reaching its highest level at 20 μM, 40 μM and 100 μM concentrations after 48 hours of drug administration, however, no higher telomerase activity was observed after 100 μM concentration.

In general, application of TAED017 at doses of 80 μM and lower appears to be safe for corneal endothelial cells in in vitro conditions. The first degenerative changes in corneal epithelial cells after 24 hours of TAED017 application were detected with a concentration of 320 μM and lower concentrations appear to be safe.

It was observed that cornea endothelial cells were more sensitive to TAED017 treatment than epithelial cells and were damaged by prolonged drug exposure starting from the early period at doses above 80 μM, endothelial cells showed significant apoptosis and lysis. TAED017 significantly induces apoptosis in HCECs with high concentrations (e.g. 160 μM and 320 μM), but it has also been shown to cause loss of cytoplasmic vacuoles, membrane integrity and cell junctions in damaged cells as a cell death pathway.

Telomerase activity in cornea endothelial and epithelial cells increased in with the TAED017 dose administered, starting at a concentration of 2 μM (100 ng/ml). It was also revealed that human corneal endothelial cells (DSMZ HCEC B4-G12) used in the study showed lower levels of telomerase activity compared to human corneal epithelial cells, while TAED017 activity was higher in corneal epithelial cells.

### Ocular Pharmacokinetics of Cycloastragenol and Corneal Barrier Passage of TAED017 Eye Drop Formulation

An active ingredient (drug) used in an eye drop formulation should pass through the corneal barrier, reach the lens and iris tissues through the anterior chamber fluid, where it will show its effectiveness. The drug is first diluted by the existing tear fluid, and most of it is drained into the nasal cavity through the nasolacrimal canal. For a drug to pass through the corneal barrier, its molecular size, lipophilic structure, water solubility and vehicle substances that will increase its ability to adhere to the corneal surface are important.

The inventors of this application aimed to research and create a 3D organotypic corneal barrier model with morphological features similar to the human corneal structure (500-550 μm thick, endothelium, interstitium and liquid air interface multilayered squamous epithelial layer) and to investigate to what extent cycloastragenol can pass this barrier at different drug concentrations. When 8 μg (50 μl/drop) of cycloastragenol eye drop formulation was applied to rabbit eyes, it was aimed to evaluate the concentrations reaching the anterior chamber fluid, lens and iris tissues at different time periods (30, 60, 120, 240 and 480 min) after application.

At 24th, 48 and 72nd hours after the addition of 4 μg and 8 μg TAED017 to the plates each containing 3D organotypic corneal barriers, the culture medium was removed from the bottom of the transwell and the drug concentrations crossing the corneal barrier were determined by LC-MS/MS analysis. Accordingly, it was observed that the amount of TAED017 crossing the corneal barrier at the 24th hour was between 0.18% and 0.25% depending on the drug concentration applied, and with repeated drug administration, this ratio was the highest 1.35% at the 48th hour and 1.89% at the 72nd hour. In terms of 3D corneal barrier crossing ability, TAED017 passage rates showed an increase at 48th hour at 4 μg concentration and followed a steady course at 72 hours. At 8 μg concentration, it was observed that TAED017 followed a parallel curve with the number of applications and reached the highest concentration passing the corneal barrier after the 3rd application.

In rabbit ocular pharmacokinetic model, 4 μg TAED017 drops (50 μl) were administered to both eyes of the rabbits twice a day for 5 days. At the end of the fifth day, blood, tear, cornea, anterior chamber fluid, iris and lens tissues were collected at 30, 60, 120, 240 and 480 minutes following the last administration of 4 μg/50 μl. According to the obtained results, significantly, cycloastragenol concentrations below 1 ng were measured in the euthanized eye tissues without drug administration at the zero hour of the study following 5 days of eye drop administration. However, the highest drug concentration was reached in all eye tissues analyzed in the 30th minute (Tmax) following the last drug administration compared to other time periods. According to the results of the drug analysis at the 30th minute, the highest drug concentrations of 215.56 ng in the corneal tissue, 4 ng in the anterior chamber and 14.12 ng in the iris were detected individually, proving that the drug passes into the corneal tissue after application to the corneal surface and reaches the intraocular tissues.

In terms of ocular pharmacokinetics, studies disclosed above determined that TAED017 eye drop formulations successfully penetrate the corneal barrier and reach the anterior chamber, lens and iris tissues. The lower drug concentration detected in the anterior chamber fluid compared to the iris tissue seems to be explained by the removal of the anterior chamber fluid as a result of its continuous circulation. On the other hand, the high concentrations of drug detected in the corneal tissue up to 60 minutes suggest that there is a continuous transfer of drug from the corneal surface to the intraocular tissues. This view is supported by the detection of drug at concentrations of 3ng and below in tears at 30 and 60 minutes. In the period following the application of the eye drops, dilution with the available tears and drainage through the nasolacrimal duct significantly decreased the drug concentration, but the highest level of drug absorbed to the corneal surface and continued to pass to the intraocular tissues was 5.39%, and the proportion of the drug that passed into the eye and showed its efficacy in the iris tissue was 0.35%.

### Specular Microscopy Results—Statistical Analysis and Methods

16-month-old male New Zealand rabbits were used in the study. In each group (n=8), one drop (50 μl) of TAED017 eye drop formulation was administered to the left eye twice a day for a total of 6 months, while the right eye was left untreated. When determining the starting age of rabbits and the duration of eye drop application (6 months), 16 months was chosen to correspond to adult human age, and the study period of 6 months was modeled as the period in which New Zealand rabbits aged 7.91 years (22.75 days for adult New Zealand rabbits, corresponding to 1 year for human age).

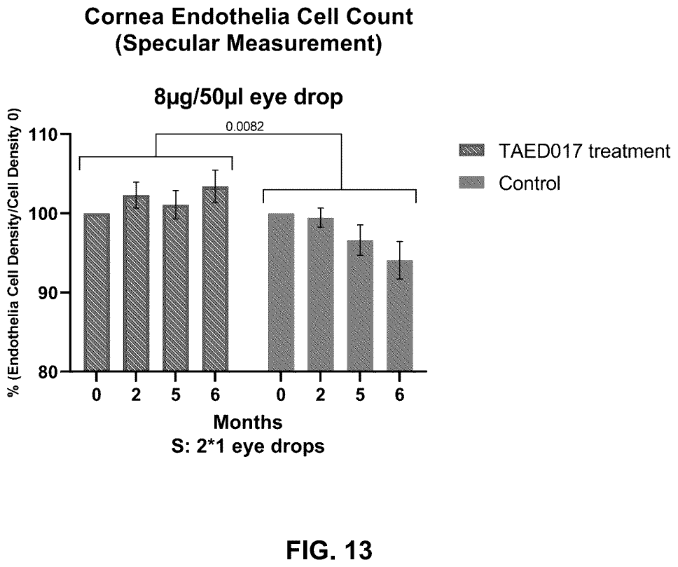

In FIGS. 12-15, data are shown as mean±standard error. The statistical evaluations are based on CD (cornea endothelia cell density) values. All CD values were corrected to the baseline measurement (month 0) of the respective rabbit and this correction was made as a percentage [(CD value/CD baseline value)*100]. GraphPad Prism 8.0.2 software was used for all statistical analyses and visualizations. The treatment of the rabbits at the respective dose was compared between the left (TAED017 treatment) and right (non-treated control) eye, both temporally and time and side dependent. For all analyses, “2-way ANOVA test for repeated measures” was used. For data sets with missing data, this analysis was set as “mixed-effect analysis” by the program. “Tukey” and “sidak” tests were used for posthoc analyses. The p values for these tests are adjusted p values.

FIG. 12 depicts cornea endothelial cell count for 4 μg/50 μl eye drop formulation group. The overall comparison between the right and left eye was performed with two way ANOVA. There was no statistically significant difference between the two groups (p=0.0706).

Multiple comparison tests were performed to determine the difference between months in each eye. While there was no significant difference between the times in TAED017 group, statistically significant differences were determined in 0-2 (p=0.0485); 0-5 (p=0.0281); 0-6 (p=0.0407) in the control group.

Multiple comparison tests were performed to see if there was a difference between the treatment and control groups in each month. While there was no statistically significant difference between the treatment and control groups in the 2nd and 5th months, the difference between the 6th months was statistically significant (p<0.05).

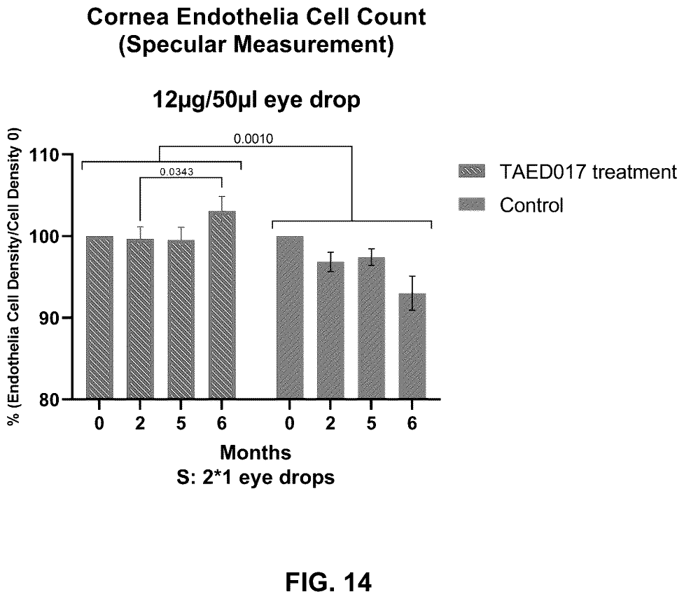

FIG. 13 depicts cornea endothelial cell count for 8 μg/50 μl eye drop formulation group. A mixed-effects analysis (ANOVA analogue test) was performed to see if there was a difference in general tendency between the right and left eye. A statistically significant difference was found between the right and left eye (p=0.0082).

There was no statistically significant difference between the right and left eye between the months. Multiple comparison tests were performed to see if there was a difference between the treatment and control groups in each month. While there was no difference between the treatment and control groups in the 2nd and 5th months, the difference between the 6th months was statistically significant.

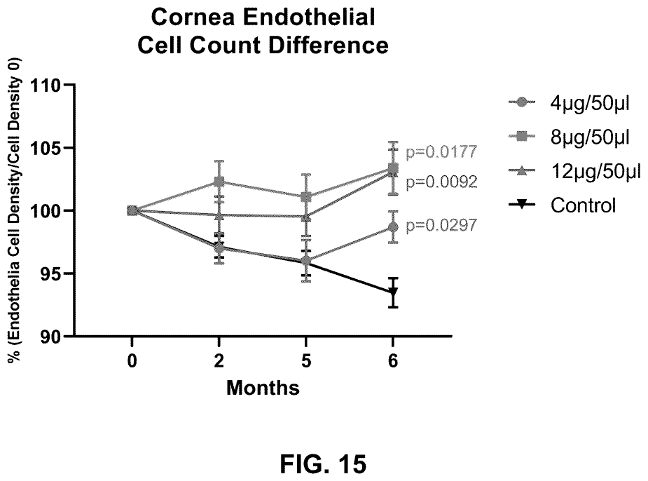

FIG. 14 depicts cornea endothelial cell count for 12 μg/50 μl eye drop formulation group. When it was examined whether there was a difference in general tendency between the right and left eye, mixed-effects analysis (ANOVA analog test) was performed instead of two-way ANOVA because the number of n was not equal. A statistically significant difference was found between the right and left eye (p=0.0010).

Multiple comparison tests were performed to determine the difference between the months in each eye. There was a statistically significant difference between the 2nd and 6th month in the TAED017 group (p=0.0343), while there was no difference between the two time periods in the control group.

Multiple comparison tests were performed to see if there was a difference between the treatment and control groups in each month. While there was no difference between the treatment and control group in the 2nd and 5th months, there was a statistically significant difference in the 6th month.

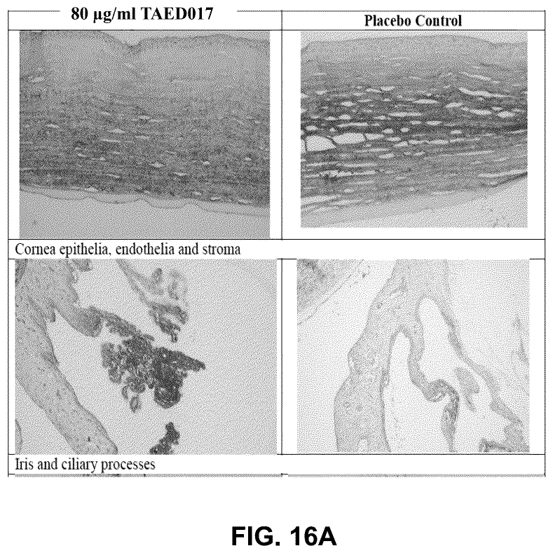

The graph of time-dependent corneal endothelial cell count changes in all dose groups treated with TAED017 eye drops is shown in FIG. 15. The arm drawn as control belongs to the average of the data belonging to the untreated right eyes of the rabbits in the three groups described above.

### Histopathology-Immuno-Histochemical Findings

In terms of telomerase reverse transcriptase (TERT) activity in the evaluated eye tissues, significantly higher expression was observed in TAED017-treated left eye cornea and iris tissues compared to untreated tissues. It was revealed that Tert activity increased in parallel with the increase in TAED017 eye drop concentration. Accordingly, the lowest Tert immunoreactivity was detected in the 80 μg group in the cornea and iris tissues, while the highest reactivity was observed in the 240 μg/ml group. Telomerase activity in the corneal epithelial layer showed cytoplasmic and nuclear staining in the 160 μg/ml and 240 μg/ml concentrations of TAED017, while activity was detected throughout all epithelial layers, especially in the 240 μg/ml TAED017 group. In corneal endothelial cells, especially in 160 μg and 240 μg/ml TAED017-treated eye tissues, a continuous layer staining was observed with a prominent and higher nature of endothelial cells compared to the placebo control group. This was characterized by weak telomerase activity in the endothelial layer with an expression of limited number of endothelial cells. While homogeneous fibril staining occurred nonspecifically in the corneal stroma, nuclear granular telomerase reactivity was observed in stromal keratinocytes in TAED017 treated groups.

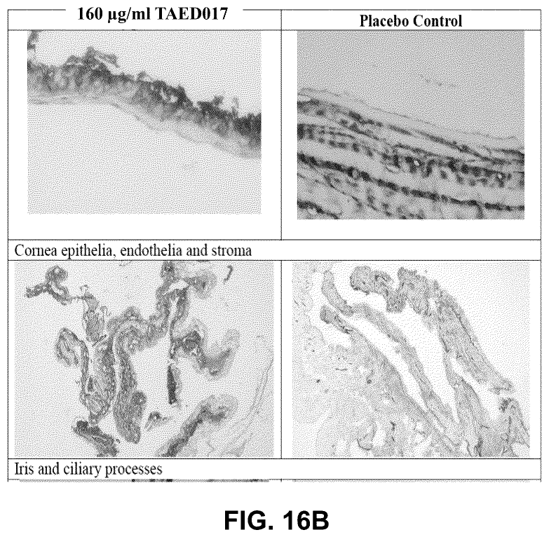

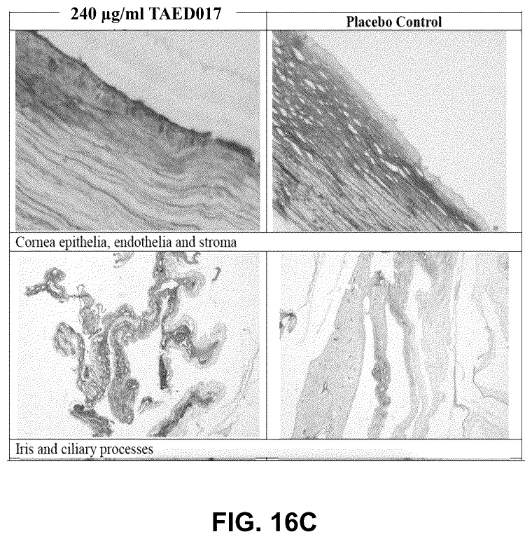

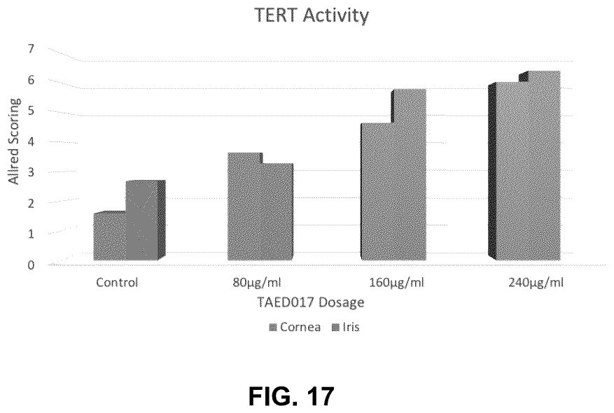

Telomerase reverse transcriptase (TERT) activity in the iris and choroidea was significantly higher in the treatment groups. Especially in eyes treated with 160 μg/ml and 240 μg/ml TAED017, regional and less severe reactions were observed in the iridocorneal angle and corpus ciliare, and diffuse and very intense immunopositivity was observed in the ciliar processes, as seen respectively in FIGS. 16B and 16C.

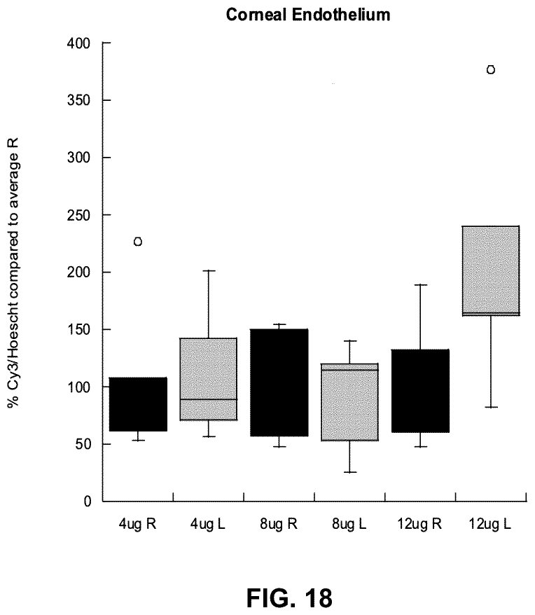

A dose-dependent increase in telomerase reverse transcriptase (TERT) activity was observed in ocular tissues treated with TAED017. Specifically, immunohistochemical analysis revealed significantly elevated TERT expression in the cornea and iris of the treated left eyes compared to untreated controls. The lowest levels of TERT immunoreactivity were detected in tissues exposed to 80 μg/ml TAED017, whereas the highest levels were observed in the 240 μg/ml treatment group (FIG. 17).

### Telomere Length Analysis/Results

To determine the mechanism by which treatment with TAED017 prevents Corneal Endothelial Cell (CEC) attrition, telomere lengths from CECs of both treated and untreated eyes were measured and compared. CEC telomere lengths were longer in eyes treated with TAED017 than in untreated animals (as shown in FIG. 18). The results shown in FIG. 18 illustrate that TAED017 treated CEC telomeres are longer than those from untreated eyes (L: treated eyes, R: non-treated control eyes).

We then evaluated whether the observed effect of telomere lengthening in CECs also occurred in other ocular tissues. Rabbit eyes were treated for six months with TAED017, (one eye of each rabbit was treated while the other eye remained untreated, as a control group). In eyes treated with TAED017, a significant increase in telomere length of corneal endothelial cells was observed.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. As such, to the extent any embodiments are described as less desirable than other embodiments or prior art implementations with respect to one or more characteristics, these embodiments are not outside the scope of the disclosure and can be desirable for particular applications.

What is claimed:

1. An ophthalmic formulation for treating, preventing and/or reducing a risk of a corneal endothelial dysfunction, wherein the ophthalmological formulation comprises a therapeutically effective amount of a telomerase activator.

2. The ophthalmic formulation of claim 1, wherein the corneal endothelial dysfunction is Fuchs' endothelial corneal dystrophy (FECD), corneal edema, bullous keratopathy (PBK), acute or chronic corneal endothelial cell (CEC) loss, posterior polymorphous corneal dystrophy (PPCD), congenital hereditary endothelial dystrophy (CHED), Iridocorneal Endothelial (ICE) Syndrome, corneal degeneration, corneal melting, or corneal ectasia, ocular trauma, or any combination thereof.

3. The ophthalmic formulation of claim 2, wherein the corneal endothelial dysfunction is Fuchs' endothelial corneal dystrophy (FECD).

4. The ophthalmic formulation of claim 1, wherein the telomerase activator comprises at least one of cycloastragenol, astragaloside I, astragaloside II, astragaloside IV, astragaloside VII (AST VII), cyclocephaloside I (CCI), cyclocanthoside E (CCE), andrographolide, resveratrol, epitalon, estrogen, omega-3 fatty acids, their derivatives or combinations thereof.

5. The ophthalmic formulation of claim 1, wherein the telomerase activator is present in a concentration of about 0.3 μg/ml to about 370 μg/ml?

6. The ophthalmic formulation of claim 1, wherein the formulation is packaged in preservative-free eye drop system, which does not include an antibacterial agent.

7. The ophthalmic formulation of claim 1, further comprising an antimicrobial agent, a chelator and an excipient.

8. The ophthalmic formulation of claim 7, wherein the antimicrobial agent contained in the ophthalmic formulation is selected from the group consisting of benzalkonium chloride, polyhexanide (PHMB, Polyhexamethylene Biguanide), chlorhexidine, EDTA, purite (stabilized oxychloro complex (SOC)), polyquad (polyquaternium-1), SofZia, or a combination thereof.

9. The ophthalmic formulation of claim 7, wherein the chelator contained in the ophthalmic formulation is selected from the group consisting of ethylenediaminetetraacetic acid disodium salt, ethylenediamine salt, ethyleneglycoltetraacetic acid salt, diethylene triamine pentacetic acid (DTPA), ethylenediamine tetramethylene phosphonic acid, citric acid, citrates, ETA, Deferoxamine, Deferasirox, and Deferiprone, or combinations thereof.

10. The ophthalmic formulation of claim 7, wherein the excipient is at least one of polyvinylpyrrolidone, ethanol, dimethyl sulfoxide, hydroxypropyl methylcellulose (HPMC), carbomer (carbopol-polyacrylic acid derivatives), sodium hyaluronate (hyaluronic acid), polyvinyl alcohol (PVA), polyethylene glycol (PEG) or propylene glycol (PPG), carboxymethylcellulose sodium-CMC, xanthan gum or combinations thereof, at a concentration of about 0.05% (w/v) to about 0.9% (w/v) or about 0.05% (v/v) to about 1% (v/v).

11. A method of preventing, treating and/or reducing a risk, or slowing progression of corneal endothelial dysfunction in a subject, the method comprising administering to the subject a formulation comprising a telomerase activator in an amount effective to prevent, treat and/or reduce a risk of the corneal endothelial dysfunction.

12. The method of claim 11, wherein the corneal endothelial dysfunction is Fuchs' endothelial corneal dystrophy (FECD), corneal edema, bullous keratopathy (PBK), acute or chronic corneal endothelial cell (CEC) loss, posterior polymorphous corneal dystrophy (PPCD), congenital hereditary endothelial dystrophy (CHED), Iridocorneal Endothelial (ICE) Syndrome, corneal degeneration, corneal melting, or corneal ectasia, ocular trauma, or any combination thereof.

13. The method of claim 11, wherein the telomerase activator comprises at least one of cycloastragenol, astragaloside I, astragaloside II, astragaloside IV, astragaloside VII (AST VII), cyclocephaloside I (CCI), cyclocanthoside E (CCE), andrographolide, eesveratrol, epitalon, estrogen, omega-3 fatty acids, their derivatives or combinations thereof.

14. The method of claim 11, wherein the formulation comprises the telomerase activator at a concentration of about 0.3 μg/ml to about 370 μg/ml.

15. The method of claim 14, wherein the formulation further comprises an antimicrobial agent, a chelator and an excipient.

16. The method of claim 15, wherein the antimicrobial agent contained in the ophthalmic formulation is selected from the group consisting of benzalkonium chloride, polyhexanide (PHMB, Polyhexamethylene Biguanide), chlorhexidine, EDTA, purite (stabilized oxychloro complex (SOC)), polyquad (polyquaternium-1), SofZia, or a combination thereof.

17. The method of claim 15, wherein the chelator contained in the ophthalmic formulation is selected from the group consisting of ethylenediaminetetraacetic acid disodium salt, ethylenediamine salt, ethyleneglycoltetraacetic acid salt, diethylene triamine pentacetic acid (DTPA), ethylenediamine tetramethylene phosphonic acid, citric acid, citrates, ETA, Deferoxamine, Deferasirox, and Deferiprone, or combinations thereof.

18. The method of claim 15, wherein the excipient is at least one of polyvinylpyrrolidone, ethanol, dimethyl sulfoxide, hydroxypropyl methylcellulose (HPMC), carbomer (carbopol-polyacrylic acid derivatives), sodium hyaluronate (hyaluronic acid), polyvinyl alcohol (PVA), polyethylene glycol (PEG) or propylene glycol (PPG), carboxymethylcellulose sodium-CMC, xanthan gum or combinations thereof, at a concentration of about 0.05% (w/v) to about 0.9% (w/v) or about 0.05% (v/v) to about 1% (v/v).

19. An eye drop formulation, an injection or topical ointment for treating, preventing and/or reducing a risk of, or slowing progression of a corneal endothelial dysfunction, wherein the eye drop formulation comprises a telomerase activator in an amount of 0.3 μg/ml to about 370 μg/ml, an antimicrobial agent, a chelator, and an excipient.

20. The eye drop formulation, injection or topical ointment of claim 19, wherein the corneal endothelial dysfunction is Fuchs' endothelial corneal dystrophy (FECD), corneal edema, bullous keratopathy (PBK), acute or chronic corneal endothelial cell (CEC) loss, posterior polymorphous corneal dystrophy (PPCD), congenital hereditary endothelial dystrophy (CHED), Iridocorneal Endothelial (ICE) Syndrome, corneal degeneration, corneal melting, or corneal ectasia, ocular trauma, or any combination thereof
